SEQUENCE LISTING (6 sequences)

SEQ ID NO: 1 - DNA, 16 bp, Artificial Sequence
  M13 (-24) Reverse Primer
aacagctatgaccatg

SEQ ID NO: 2 - DNA, 24 bp, Artificial Sequence
  M13 (-48) Reverse Primer
agcggataacaatttcacacagga

SEQ ID NO: 3 - DNA, 17 bp, Artificial Sequence
  M13 (-20) Forward Primer
gtaaaacgacggccagt

SEQ ID NO: 4 - DNA, 17 bp, Artificial Sequence
  M13 (-40) Forward Primer
gttttcccagtcacgac

SEQ ID NO: 5 - DNA, 20 bp, Artificial Sequence
  T3 Primer
aattaaccctcactaaaggg

SEQ ID NO: 6 - DNA, 22 bp, Artificial Sequence
  T7 Primer
gtaatacgactcactatagggc

(19) United States
(12) Patent Application Publication
(10) Pub. No.: US 2003/0180735 A1
(43) Pub. Date: Sep. 25, 2003

(54) Screening method

(76) Inventors: Tom Cotter, Cork (IE); Ian Hayes, Cork (IE)

Correspondence Address: David R Marsh, Arnold & Porter, IP Docketing Dept., Room 1126B, 555 12th Street N W, Washington, DC, 20004-1206, US

(21) Appl. No.: 10/168,448

(22) PCT Filed: Dec. 21, 2000
(86) PCT No.: PCT/IB00/02054

(30) Foreign Application Priority Data
Dec. 21, 1999 (GB) 9930255.6

(51) Int. Cl. C12Q 1/68; G01N 33/574

(52) U.S. Cl. 435/6; 435/7.23

(57) ABSTRACT

The invention relates to a system for modelling ROS-induced apoptosis in a cell, said system comprising: a) the provision of a population of cells; b) the exposure of said cell population to one or more stimuli which lead the cell population to undergo ROS-induced apoptosis; c) optionally, the exposure of said cell population to one or more inhibitors of ROS-induced apoptosis; d) the analysis of gene expression in the cell population; and e) the assessment of the onset of apoptosis in said cell population; and methods for identifying genes associated with ROS-induced apoptosis using such a system.

The present invention relates to a method for identifying genes which are involved in the modulation of apoptosis in cells. In particular, the invention relates to a novel method for modulating the expression of gene products involved in the early stages of apoptosis, by a redox mechanism, and their subsequent identification and characterisation, before the cell has made a commitment to the apoptotic fate.

Programmed cell death or apoptosis is a genetically programmed process by which cells die under both physiological and a variety of pathological conditions (Kerr et al, Br. J. Cancer, 26, 239-257, 1972). It serves as the counter-balancing force to mitosis during adult life and is a major contributor to the sculpting of physiological structures during the many processes of development (Wyllie et al, Int. Rev. Cytol, 68, 251-305, 1980). It is characterised by a number of well defined biochemical hallmarks. These include DNA fragmentation, caused by the activation of an endogenous endonuclease enzyme (Wyllie, Nature, 284, 555-556, 1980; Enari et al., Nature, 391, 43-50, 1998). The result is a DNA ladder pattern which can be readily visualised in agarose cells. Coupled with DNA fragmentation is cell shrinkage (Wesselbory et al., Cell Immunol. 148, 234-41, 1993) where water is actively extruded from the cell. The apoptotic cell then undergoes fragmentation into apoptotic bodies which are engulfed by neighbouring cells or cells of the reticulo-enothelial system.

A second well defined characteristic is the exposure of the phospholipid phosphatidylserine to the outside surface of the plasma membrane of the cell as it undergoes apoptosis (Fadok et al., J Immunol. 148, 2207-16, 1992). Normally this lipid is located on the inner side of the membrane lipid bilayer. The underlying mechanism responsible for this lipid flipping is poorly understood at present. Its expression serves as a signal for the recognition and phagocytosis of the apoptotic cell (Fadok et al., J Immunol. 148, 2207-16, 1992)

The signal transduction system of apoptosis is relatively poorly understood at present, but a series of enzymes called caspases have been shown over the last few years to be involved in different phases of apoptosis, in particular in the propagation and execution phases of the process (Samali et al., Cell Death Differ. 6, 495-6, 1999). For example, caspase 3 is involved in the activation of CAD, an endonuclease involved in the cleavage of DNA to yield the hallmark DNA ladder of apoptosis described above (Mcllroy et al., Cell Death Differ. 6, 495-6, 1999). This caspase cleaves ICAD, a natural inhibitor of the CAD endonuclease. Other caspases are involved in the propagation and execution phase of the process, an example of which is caspase 8. Not all incidences of apoptosis involve these enzymes and there is evidence that under conditions of high oxidative stress these cysteine containing proteases are inactivated. To date 14 caspases have been identified as playing a role in the process (Alnemri et al., J Cell Biochem. 64, 33-42, 1997).

Under normal physiological conditions apoptosis is tightly regulated. However, there are a number of diseases where the process becomes deregulated, leading to a particular pathology. Examples of where apoptosis is retarded or inhibited include some types of tumour development, a number of inflammatory conditions such as adult respiratory distress syndrome (ARbS) and other related conditions (Matute-Bello et al, Am J Respir Crit Care Med. 56, 1969-77, 1997). Inappropriate or excessive apoptosis occurs under conditions of ischaemia (stroke, myocardial infarction, etc) Linnik et al., Blood. 80, 1750-7, 1992, Gorman et al., J Neurol Sci. 139, 45-52, 1996) a series of neurodegenerative conditions, myelosuppression (Mori et al, Blood. 92, 101-7, 1998) following chemotherapy or irradiation (Lotem et al., Blood. 80, 1750-7, 1992) and a significant number of other diseases where cell death is a key feature of the pathology.

A key event in the initiation and propagation of apoptosis is the generation of reactive oxygen species (ROS) (McGowan et al., Exp Cell Res, 238, 248-56, 1998). These species include hydrogen peroxide, superoxide and the hydroxyl radicals. They can be generated either at the level of the mitochondrion, where any disruption of the respiratory chain can lead to their production or via a number of enzyme reactions such as NADPH oxidase (Nauseef et al., Proc Assoc Am Physicians, 111, 373-82, 1999). This enzyme is particularly active in the neutrophil. Such molecules cause oxidative damage not only to cellular structures, but may also act to initiate the expression apoptosis regulating genes. In mammalian cells the physiological role for such ROS molecules is far less well characterised than that of other related molecules such as nitric oxide (NO). In relation to the involvement of NO in apoptosis the published literature is unclear, with examples of NO both driving and inhibiting apoptosis (Brune et al., Cell Death Differ. 1999 10,969-975, 1999). There is an increasing volume of evidence for a ROS role in driving apoptosis, but the mechanisms of this is not understood at present (Hildeman et al., Immunity. 10,735-44, 1999, Gorman et al., J Neurol Sci. 139, 45-52, 1996).

Studies over the past 5 years have demonstrated that a variety of cytokines, growth factors and agents that induce apoptosis can lead to the generation of ROS. These studies have suggested that ROS may act as second messengers in signal transduction pathways in the context of cytokine/growth factor stimulation of cells. Other more recent studies have indicated that they may also activate unique pathways. The specific targets of ROS generated intracellularly are largely unknown at present, but it is known that the addition of hydrogen peroxide or other ROS generators to cells in culture leads to the activation of the transcription factor Nf/kB (Schreck et al., EMBO J 10, 2247-2258, 1991). This in turn controls the expression of a series of genes involved in a variety of cellular functions. Other targets of ROS include the activation of the mitogen activated protein kinase (MAPK) which is known to be involved in the regulation of cell proliferation (Kamata et al., J. Biol. Chem, 271, 33018-33025).

A cell has the ability to produce ROS at a number of different sites. In relation to signal transduction events it is still unclear where the source of ROS is within the cell. There are a number of potential enzyme systems capable of ROS generation. Perhaps the best documented one, particularly in neutrophils and other phagocytic cells, is NADPH oxidase. Studies using inhibitors of this enzyme such as DPI suggest that this enzyme is also involved in the generation of ROS in non phagocytic cells (Griendling et al., Circulation, 74, 1141-1148, 1994). Mitochondria play a key role in apoptosis and are also a major site of ROS generation. The loss of mitochondrial membrane potential is coupled to the release of cytochrome C and this in turn has two effects. The first is the generation of ROS, since the respiratory chain is disrupted by the removal of cytochrome C. The second is the cleavage of cellular DNA through a series of cytochrome C mediated caspase activation steps, which is an end point of the apoptosis process.

It has been suggested that ROS are involved in p53 mediated apoptosis (Johnson et al., Proc. Natl. Acad. Sci. USA, 93, 11848-11852, 1997). Cells generated to over-express p53 undergo apoptosis, accompanied by ROS production and this can be blocked by anti-oxidants (Polyak et al., Nature, 389, 300-305, 1997). There are a number of other examples where ROS production is closely associated with the initiation and propagation of apoptosis. However, the mechanism of ROS activity in apoptosis is not understood.

Cells in their struggle for survival have developed a series of defence mechanisms to counter the death inducing effects of ROS. Such defences include the biological anti-oxidant glutathione (GSH) which has the ability to counter the effects of low levels of ROS and thus maintain a redox balance in the cell. The ability of a cell to respond to oxidative stress by the upregulation of redox modulated gene may play a key part in a cells survival strategy.

A series of enzymes involved in maintaining the redox balance within a cell contribute to the ability of that cell to survive in the presence of elevated ROS levels. Such enzymes include catalase and superoxide dismutase which work to reduce the oxidative stress in cells. In addition to redox modulating enzyme several other proteins most notably BcI-2 are thought to mediate their anti-apoptotic effects via an anti-oxidant process (Hockenberry et al., Cell. 1993 75:241-51). The precise mechanism by which BcI-2 mediates its effects are still not quite defined. Other proteins such as members of the heat-shock family have also been demonstrated to protect cells from undergoing apoptosis in a pro-oxidant environment (Creagh et al., Leukaemia. 2000 (7):1161-73. The redox sensitive transcription factor NF-kB is also known to induce the expression of a series of genes (some known and others yet to be discovered) which modulate the cells ability to undergo apoptosis.

Chemotherapeutic agents used in the treatment of cancer kill the tumour cells by the induction of apoptosis and in the case of some chemotherapeutic agents this appears to be mediated through the generation of ROS. Such agents include cisplatin and alkylating agents. Thus an understanding of how these and other related agents affect the redox balance of the tumour cells would be invaluable in developing strategies for enhancing the killing of target cells. The use of agents to increase the oxidative stress in tumour cells may sensitise tumour cells to ROS generating chemotherapeutic agents. Thus the identification of novel redox modulating proteins may provide us with valuable targets to sensitise tumour cells to currently used chemotherapeutic agents. Support for this concept has come from several recent publications most notable a paper in Nature (Huang et al., 407,390-395, 2000) where 2-methoxyoestradiol (2-ME) an oestrogen derivative inhibited the enzyme superoxide dismutase thus increasing the oxidative stress in leukaemia cells. The effect of this was to sensitise cells to apoptosis because the generation of ROS which would be normally counteracted by SOD activity could not take place. The elevated metabolism of tumour cells and as a result increased generation of ROS may offer a selectivity of treatment by interfering with the redox balance in tumour cells. In addition Voehringer et al in PNAS 97, 2680-2685, 2000 demonstrated that the ROS generating and apoptosis inducing insult of UV irradiation, induced the expression of several genes involved in the modulation of the redox balance of cells. Included were genes that code for the proteins CD53 and fructose-1,6 bisphosphatase, both of which can lead to an increase in the production of glutathione in the cell.

### SUMMARY OF THE INVENTION

The control of apoptosis represents a significant therapeutic target, since many diseases are due to defects in this process. There is an acute need to identify the genes that regulate this process. In other words, if one identifies a gene that drives cell apoptosis, then this gene/gene product or its function can be blocked by a drug and apoptosis inhibited. To-date many of the genes found have certain fundamental flaws e.g. they act late in the process, after the cell has committed to a death programme, or they are ubiquitous, that is they are not restricted to a particular cell type. The ideal target to control apoptosis act early in the process and are restricted to a particular cell type.

Furthermore, many physiological factors prevent cell apoptosis. For example, cytokines or growth such as GM-CSF inhibit death through apoptosis. Identification of a gene that prevents apoptosis allows this gene/gene product or its function to be blocked by a drug and apoptosis allowed to occur.

We hypothesised that ROS/REDOX is a fundamental ‘early’ physiological signal in many cells, if not all cells, for the induction of apoptosis (programmed cell death). By ‘early’ is meant before the cell has ‘committed’ to activation of the apoptosis programme. This signal acts through a signal transduction pathway and is associated with significant changes, or patterns of changes, in gene expression in the cell. If model discovery assays are configured which target these ‘early’ induction events occurring in apoptosis by a common physiological stimulus it is possible to identify the key genes to control apoptosis regulation.

Furthermore, many chemotherapeutic drugs act through an ROS/REDOX mechanism of apoptosis. By identifying genes whose products mediate a cell's defence against ROS/REDOX-mediated apoptosis it is possible to target this gene and thus reduce a cancer cell's resistance to chemotherapeutic drug treatment.

Genes regulated in these models following induction of apoptosis by ROS (either endogenous, or by exogenous addition) include genes that 1) initiate the process of apoptosis (pro-apoptotic genes) and thus represent therapeutic targets, 2) make up aspects of the ROS signal cascade and thus represent therapeutic targets, and 3) are involved in the cells defence mechanisms aimed at preventing apoptosis (anti-apoptotic) and thus represent therapeutic targets.

If apoptosis is then inhibited in these models, either by a physiological cytokine e.g. GM-CSF, or by some inhibitor of the ROS signal, the changes in gene expression also include genes that 1) initiate the process of apoptosis (pro-apoptotic genes) and thus represent therapeutic targets, 2) make up aspects of the ROS signal cascade and thus represent therapeutic targets, and 3) are involved in the cells defence mechanisms aimed at preventing apoptosis (anti-apoptotic) and thus represent therapeutic targets.

These different models may be combined to identify those genes most closely associated with apoptosis. For example, a gene whose expression decreases during apoptosis, but increases during cytokine mediated survival is positively correlated with the process. For example, a gene that is increased in two or more independent models both of which act through an ROS/REDOX mechanism is positively correlated with the process.

We have further designed a ‘genomics strategy’ that allows us to identify these ‘key’ apoptosis/defence genes using our models.

Using this genomic strategy we have demonstrated the foregoing and the utility of these models and this strategy. We have proven this by reference to known genes involved in apoptosis and cell response to ROS. These and also the large number of novel genes, similarly identified, represent potential therapeutic targets identified by this approach.

We have determined that production of intracellular ROS is causally or consequentially connected with the modulation of early transcription and/or translation, and/or post-translational modification in cells of genes which control the progression of the cell towards apoptosis. Unlike caspases and other genes known to be involved in apoptosis, which generally act at the execution stage of apoptosis and are only activated once the cell is committed to the apoptotic fate, the genes whose expression is modulated during or after ROS exposure are required for induction of apoptosis, before the cell has made a commitment to die. Accordingly, regulation of the expression of ROS-associated genes provides a mechanism by which the entry of the cell into the apoptotic process may be induced or prevented.

In a first aspect, the invention provides a method for identifying a gene product which modulates the transition of a cell between a non-apoptotic state and an apoptotic state, comprising the steps of:

a) determining the level(s) of expression of one or more gene product(s) in a cell to establish a reference expression level;

b) exposing the cell to one or more stimuli which induce the production of intracellular ROS;

c) monitoring the level(s) of expression of said one or more gene product(s) in the cell; and

d) identifying gene product(s) whose expression has been increased, decreased or modified as a result of ROS exposure.

The intracellular ROS concentration may be increased by administering exogenous ROS, including O₂⁻ and H₂O₂, or by inducing endogenous ROS production in the cell. References to ROS herein are equivalent to references to changes in REDOX in the cell. Moreover, ROS concentration may be modulated through culture in a medium containing serum, exposure to cisplatin. exposure to UV irradiation and exposure to TNFα and cycloheximide.

The method of the invention is applicable to the discovery of gene products, and the genes encoding them, which are involved in apoptosis. The gene products may be polypeptides and/or RNAs. “Polypeptide” herein refers to any peptide comprising two or more amino acids, whether comprising a single domain or multiple domains, and includes multi-subunit proteins, which are cellular gene products. RNA gene products include ribozymes, antisense RNA molecules and/or mRNA molecules. Advantageously, the gene products are natural gene products, that is they are encoded by naturally-occurring genes in the cell being investigated and are assembled in the cell using natural components such as amino acids or nucleotides. However, the invention also encompasses screening for gene products encoded by genes which are not endogenous to the cell being investigated. Such genes may be, for example, heterologous genes from other cells or organisms, artificial genes encoding polypeptides comprising domains from different sources or composite RNA molecules, and wholly or partially randomised genes encoding repertoires of polypeptide or nucleic acid gene products.

It has been found that exposure of the cell to ROS leads to induction of apoptosis in the cell at a different rate than occurs under identical conditions but in the absence of ROS. The assay of the invention may be configured to identify gene products which accelerate or retard the induction of apoptosis. Advantageously, the assay detects gene products which accelerate the induction of apoptosis.

Levels of gene expression may be determined in any appropriate manner. Preferably, the invention comprises the measurement of protein production by mRNA translation, and is configured to detect increases or decreases in the rate or amount of mRNA translation. The invention may also be configured to detect changes in post-translational processing of polypeptides or post-transcriptional modification of nucleic acids. For example, the invention may be configured to detect the phosphorylation of polypeptides, the cleavage of polypeptides or alternative splicing of RNA, and the like. Levels of expression of gene products such as polypeptides, as well as their post-translational modification, may be detected using proprietary protein assays or techniques such as 2D polyacrylamide gel electrophoresis. Polypeptide or nucleic acid populations may be assessed individually, or together, in order to identify candidate gene products.

Advantageously, expression levels are assessed by measuring gene transcription. This is preferably carried out by measuring the rate and/or amount of specific mRNA production in the cell. A preferred embodiment of this aspect of the invention involves the use of arrayed oligonucleotide probes capable of hybridising to mRNA populations. Differences in hybridisation patterns of different mRNA populations may be used to identify genes which are differentially expressed in the two populations. The arrayed oligonucleotide probes are advantageously derived from cDNA or EST libraries, and represent genes which are expressed by the cells under investigation.

As used herein, the terms “oligonucleotide” and “polynucleotide” are equivalent, and imply no limitation as to maximum or minimum length.

A reference expression level, once established for a given cell type, may be used for repeated screens, thus facilitating the performance of repeated rounds of screening.

Cells useful in the method of the invention may be from any source, for example from primary cultures, from established cell lines, in organ culture or in vivo. Cell lines useful in the invention include fibroblast cell lines, carcinoma cell lines such as neuroblastoma cell lines and cell lines of haematopoietic origin. Preferred are primary cultures of neutrophils or cells having neutrophil characteristics, for example HL-60 cells, or Ntera-II cells. HeLa cells and other cells may also be used.

The ROS may be supplied by adding exogenous ROS to the cells, for example in the form of O₂⁻ or H₂O₂, or by inducing endogenous ROS production by methods such as those described above. In order to increase the length of the window between ROS addition and the onset of apoptosis, which is the time period during which modulation of the expression of genes involved in apoptosis induction is detectable, an inhibitor of apoptosis may be added to the cells. An example of a suitable inhibitor is GM-CSF.

A number of methods are known in the art for monitoring the onset of apoptosis. These include morphological analysis, DNA ladder formation, externalisation of membrane phospholipid phosphatidyl serine and caspase activation analysis. The ability of ROS to induce apoptosis is preferably confirmed by monitoring the onset thereof according to one or more of the above methods. Moreover, ROS inhibitors, such as NAC, PDTC and DPI may be used to further substantiate the role or ROS.

In a further aspect, the invention relates to the use of ROS to induce the expression of gene products which modulate the transition of a cell between an apoptotic state and a non-apoptotic state. Preferably, the use according to the invention further comprises identification of the gene products and/or the genes encoding the gene products.

In a still further aspect, the invention may be configured to detect changes in gene expression which occur as a result of exposure to cells to apoptosis inhibitors such as GM-CSF. We have determined that GM-CSF is responsible for the induction of the expression of anti-apoptotic genes, including genes which combat the action of ROS in inducing apoptosis in the cell.

Accordingly, the invention provides a method for identifying a gene product which modulates the transition of a cell between a non-apoptotic state and an apoptotic state, comprising the steps of:

a) determining the level(s) of expression of one or more gene product(s) in a cell to establish a reference expression level;

b) exposing the cell to an inhibitor of apoptosis and/or ROS activity;

c) exposing the cell to one or more stimuli which induce the production of intracellular ROS;

d) monitoring the level(s) of expression of said one or more gene product(s) in the cell; and

e) identifying gene product(s) whose expression has been increased, decreased or modified as a result of ROS exposure in the presence or absence of the apoptosis inhibitor.

The methods provided by the invention may be configured together or separately to provide a system for identifying genes and gene products which are responsible for the modulation of the onset of apoptosis in cells.

Thus, the invention provides a system for modelling ROS-induced apoptosis in a cell, said system comprising:

a) the provision of a population of cells;

b) the exposure of said cell population to one or more stimuli which lead the cell population to undergo ROS-induced apoptosis;

c) optionally, the exposure of said cell population to one or more inhibitors of ROS-induced apoptosis;

d) the analysis of gene expression in the cell population; and

e) the assessment of the onset of apoptosis in said cell population.

### BRIEF DESCRIPTION OF THE FIGURES

FIG. 1. Spontaneous apoptosis in the neutrophil induces caspase activity. Neutrophils are isolated from donor blood and kept in culture for the indicated time points. Following lysis of the cells, the supernatant fraction is examined for caspase activity using the CaspACE kit. Protein levels in the supernatant are determined by micro protein quantitative assay. (Sigma).

FIG. 2: Microscopic examination versus cell shrinkage as determined by Forward Scatter Parameters for analysis of neutrophil apoptosis. Duplicate samples are compared for the onset of apoptosis by either making cytospins and examining morphological changes as described in FIG. 1 or acquiring samples into FacScan and analysing for changes in Forward Scatter Paramers (FSc). As cells shrink their FSc is reduced and this is taken as a characteristic of apoptosis. From the graph it can be seen that over time, the number of cells with reduced FSc increases and this correlates with the number of cells undergoing apoptosis as determined by microscopic morphology.

FIG. 3: % neutrophils positive for hydrogen peroxide (H₂O₂) production. Isolated neutrophils are cultured for the indicated time period. One hour prior to acquisition, cells are incubated with 5 μM DCFA/DA. The percentage of cells positive for hydrogen peroxide production is determined by examining increased fluorescence (FL1) above background.

FIG. 4: Inhibition of NADPH oxidase can decrease the number of cells undergoing apoptosis. Neutrophils are cultured @2×106/ml in a 24 well plate. Following 6 hours of culture, Forward Scatter (FSc) parameters are examined following acquisition by a FacScan. Cell shrinkage is determined by the percentage of cells with reduced FSc compared with cellular FSc parameters at t=0 h.

FIG. 5: Global gene expression regulation during neutrophil spontaneous apoptosis. RNA is extracted from primary human neutrophils undergoing spontaneous apoptosis, 2, 3, 4, 5 and 6 hours post-isolation and used as probe for human LifeGrid microarray filters. Cluster analysis is performed and a hierarchical tree view depicted for those genes regulated >2-fold up (red) or down (green) wrt. Control time zero cells.

FIG. 6: Differential gene expression of novel DNA clones, isolated by SSH, during neutrophil spontaneous apoptosis. Figure shows a ‘virtual northern blot’ with clone 1053, an EST having no homologues in the public EST database. Virtual northern blots are prepared by standard procedures using SMART cDNA derived with the Clontech SMART cDNA kit as per manufacturers instructions. Clone 1053 that is expressed in neutrophils (time zero control; T0) is down-regulated during spontaneous neutrophil apoptosis at 2 and 4 hours post-isolation, respectively (T2 and T4).

FIG. 7: Differential gene expression of DNA clone, isolated by SSH, during neutrophil spontaneous apoptosis. Figure shows a ‘virtual northern blot’ with clone 1052, an EST which shares sequence identity with the protein tyrosine phosphatase. Virtual northern blots are prepared by standard procedures using SMART cDNA derived with the Clontech SMART cDNA kit as per manufacturers instructions. Clone 1052 that is expressed in neutrophils (time zero control; T0) is down-regulated during spontaneous neutrophil apoptosis at 2, 4 and 24 hours post-isolation, respectively (T2, T4 and T24).

FIG. 8: Addition of GM-CSF inhibits apoptosis: Neutrophils are incubated with the indicated amounts of GM-CSF immediately post isolation and further cultured overnight at 37° C. The following morning, viability is measured using the MTT assay as previously described. Cell viability is directly related to the colour change at 570 nm. The graph shows that maximal survival is obtained using either 5 or 50 units/ml of GM-CSF and that survival is dose dependent on amounts below this level.

FIG. 9. GM-CSF mediates survival in a temporal fashion. GM-CSF (50U/ml) is added to isolated neutrophils at the indicated times and survival measured by MTT. Cell viability is directly related to the colour change at 570 nm. Survival is conferred on neutrophils by GM-CSF when additions are made up to 5 hours post isolation. GM-CSF added 20 post isolation is not sufficient to mediate survival indicating that the cells have passed their commitment to die point.

FIG. 10. Immediate addition of anti-GM-CSF can ameliorate survival. Anti GM-CSF (10 μg/ml) is added to neutrophil cultures containing (5 u/ml) GM-CSF at the indicated times. Survival is measured by MTT. Cell viability is directly related to the colour change at 570 nm. For GM-CSF to increase full survival of neutrophils, addition of the neutralising antibody had to be delayed for 3 hours.

FIG. 11: GM-CSF mediates survival independent of glutathione. Neutrophils (2×10⁴) are cultured +/− GM-CSF (5U/ml) and +/− BSO at the indicated concentrations. Following overnight culture, cell viability is measured using the MTT assay and optical density is read @570 nm. Colour intensity is directly proportional to cell viability. The figure demonstrates that GM-CSF can enhance cell viability without the necessity of the cell to replenish intracellular glutathione, since GM-CSF also mediates survival in the presence of the γ-glutamylcysteine synthase inhibitor BSO.

FIG. 12. Expression of Glutathione Peroxidase and γ-glutamylcysteine synthase. RNA from PBMC and neutrophils are isolated “in house” while all other RNA is commercial (Clontech). Levels of expression are determined by hybridisation of the indicated tissue RNA to the Incyte LifeGrid microarray. As can be seen that there is a large expression range between the various tissues with the cerebellum having highest levels and the neutrophil the lowest.

FIG. 13. Cluster analysis of the effect of GM-CSF on neutrophil spontaneous apoptosis. Each column represents the expression of that gene at the indicated time points following GM-CSF exposure. Red indicates increased expression and green indicates decreased gene expression. Horizontal line length in the dendrogram indicates correlation, with shorter lines indicating greater similarity and longer lines representing more divergent patterns. There is a rapid decrease in gene expression for a number of genes as short as 2 hours post GM-CSF treatment. The number of genes that increased expression grew over the 6 hour study period. Of note is the cluster of genes that are up regulated between 2 to 4 hours post treatment (Inset). Included in these genes are genes associated with survival including BCl2-A1 and Mitochondrial superoxide dismutase. This cluster is therefore referred to as the survival cluster.

FIG. 14: A Schematic of intracellular events that control the oxidative environment within the cell. Superoxides are dismutated to H₂O₂ which if not converted to H₂O and O₂ by catalase might complex with nitric oxide to form toxic peroxynitrite. B Graphs showing the temporal regulation of (i) Mn SOD, (ii) Catalase and (iii) ferritin in control and GM-CSF treated neutrophils over a 6 hour period. Of interest is the fact that GM-CSF induced a large amount of transcription of Mn SOD and Ferritin at t=4 h while simultaneously inhibiting the down-regulation of catalase.

FIG. 15. GM-CSF enhances transcription of enzymes favouring the pentose phosphate pathway. RNA is isolated from neutrophils following culture for 6 hours +/−GM-CSF (5U/ml). RNA is hybridised to Incyte “LifeGrid” microarray and results are analysed as previously described. The presence of the bifunctional enzyme phosphofructokinase-2 (PFK-2)/fructose 2,6-bisphosphatase (FBPase-2) within the survival cluster as previously described, encouraged further investigation of other enzymes involved in regulation of gluconeogenesis and glycolysis. Transcription of fructose 1,6 bisphosphatase increased over the 6 hour period while levels of phosphofructokinase 1 decreased, thus inhibiting glycolysis with fructose 6 phosphate being available for NADPH generation via the pentose phosphate pathway.

FIG. 16: Effect of cisplatin on HeLa cells. HeLa cells are treated with the indicated concentrations of cisplatin for 18 hours before apoptosis is analysed by either MTT or crystal violet staining. Both methods of analyses demonstrate the dose responsive cytotoxicity of the cells towards cisplatin

FIG. 17: Cisplatin treatment induces DNA laddering in HeLa cells. HeLa cells are treated for 24 h with the indicated doses of cisplatin (μg/ml). DNA is then isolated (as previously described) and separated on a 2% agarose gel. Note the DNA laddering in all samples that received cisplatin, in contrast to the singular heavy band seen in control cells.

FIG. 18: Modulation of Cisplatin cytotoxicity by altering the redox environment. HeLa cells (2×10⁴) are exposed to cisplatin (10 μg/ml) and either NAC (A) or BSO (B). As can be seen by increasing intracellular levels of glutathione with NAC, the cell is desensitised towards cisplatin. In contrast, by decreasing the cells ability to replenish intracellular glutathione by inhibiting γ-glutamylcysteine synthase with BSO, dramatically sensitises HeLa cell towards cisplatin.

FIG. 19: Cluster analysis of the effect of cisplatin (10 μg/ml) on HeLa apoptosis. Each column represents the expression of that gene (measured using human LifeGrid microarrays) at the indicated time points following cisplatin exposure; fold change relative to a time zero control. Red indicates increased expression and green indicates decreased gene expression. Horizontal line length in the dendrogram indicates correlation, with shorter lines indicating greater similarity and longer lines representing more divergent patterns.

FIG. 20: Cluster analysis of the effects of cisplatin (10 μg/ml) and UV-irradiation on HeLa apoptosis. Each column represents the expression of that gene (measured using human LifeGrid microarrays) at the indicated time points following either cisplatin exposure or UV-irradiation (left to right; cisplatin 2 h and 4 h, UV-irradiation 2 h, 4 h and 6 h); fold change relative to a time zero control. Red indicates increased expression and green indicates decreased gene expression. Horizontal line length in the dendrogram indicates correlation, with shorter lines indicating greater similarity and longer lines representing more divergent patterns. The section bordered by a yellow box is expanded to the right and shows a cluster of genes that are up-regulated ‘early’, both following exposure to UV-irradiation and following exposure to cisplatin.

FIG. 21: Effect of UV exposure on HeLa cells. (A) Adherent HeLa cells are exposed to UV irradiation using a UV crosslinker, before being returned to culture for a further 24 hours. Cell viability is measured using a crystal violet stain and read at 570 nm. Cell viability is proportional to colour intensity. (B) DNA fragmentation following UV insult. DNA is isolated from 5×10⁶ HeLa cells 24 h post UV insult, as previously described and run on a 2% agarose gel. The DNA fragmentation is evident in samples exposed to UV for varying lengths of time 2,3, and 4 mins (B,C,D respectively) and is indicative of apoptosis, thus confirming that UV exposure causes HeLa cells to undergo apoptosis in contrast to control cells (A).

FIG. 22: TNFα induces apoptosis in HeLa cells. HeLa cells are treated with a combination of cycloheximide (5 μg/ml) and the indicated doses of TNFα overnight. HeLa apoptosis is measured by staining the remaining adherent cells with crystal violet and reading the optical densities at 570 nm.

FIG. 23: TNFα induces apoptosis in HeLa cells. HeLa cells are treated with a combination of cycloheximide (5 μg/ml) and the indicated doses of TNFα overnight. HeLa apoptosis is measured DNA fragmentation following separation on a 2% agarose gel. DNA fragmentation is seen in all samples treated with TNFα.

FIG. 24: Cluster analysis of the effect of TNFalpha/cycloheximide on HeLa apoptosis. Each column represents the expression of that gene (measured using human LifeGrid microarrays) at the indicated time points following cisplatin exposure; fold change relative to a time zero control. Red indicates increased expression and green indicates decreased gene expression. Horizontal line length in the dendrogram indicates correlation, with shorter lines indicating greater similarity and longer lines representing more divergent patterns.

FIG. 25: H₂O₂ exposure induces apoptosis. HL60 cells were prelabelled with BrdU for 2 hours and then exposed to H₂O₂. At the indicated time points, cells were lysed and the amount of BrdU labelled DNA in lysates were measured as an indicator of apoptosis.

FIG. 26: Treatment of HeLa cells with H₂O₂ induces apoptosis. Adherent HeLa cells were treated with the indicated doses of hydrogen peroxide and cell viability was determined by crystal violet staining.

FIG. 27: Extract from a ‘LifeGrid’ cDNA array hybridised with radiolabelled probe from neutrophil mRNA. The image is captured using a STORM phosphoimager (Molecular Dynamics).

FIG. 28: Screen-shot of a ‘LifeGrid’ microarray gel image undergoing Arrayvision analysis. The spots are detected using a predefined template that is manually positioned over the image. Spot density values are then calculated automatically.

FIG. 29: Cluster analysis of fold change. Neutrophils are treated with GM-CSF for 2, 4 and 6 hours post-isolation. Gene expression data captured from ‘LifeGrid’ microarrays is manipulated to calculated fold changes as compared to a time zero hour control. Fold change data is subjected to cluster analysis and represented as a hierarchical tree view. Up-regulation is depicted as red and down-regulation is depicted as green.

FIG. 30: Cluster analysis of combined code. Neutrophils are treated with GM-CSF for 2, 4 and 6 hours post-isolation. Gene expression data captured from ‘LifeGrid’ microarrays is manipulated to calculated combined codes to illustrate fold change as compared to a time zero hour control. Fold change data is subjected to cluster analysis and represented as a hierarchical tree view. Up-regulation is depicted as red and down-regulation is depicted as green.

FIG. 31: Use of microarray to confirm differential expression of SSH clones. Replicate microarrays (Filters prepared using the 96-pin replicator as described) are constructed from clones derived by suppression subtractive hybridisation; in this case a subtraction between control time zero neutrophils and neutrophils treated for 2 hours with GM-CSF. Each microarray is then probed with radiolabelled probes generated using mRNA from neutrophils treated with either GM-CSF (panel A) or control (panel B). Differential hybridisation is confirmed (compare spot indicated by arrow in panel A, with spot indicated by arrow in panel B) and false positives eliminated.

FIG. 32: Example of a microarray spotted by Genescreen, using DNA sequences isolated by SSH. Microarray is hybridised to radiolabelled probe from HL60 mRNA.

### BRIEF DESCRIPTION OF THE TABLES

Table 1: % neutrophils undergoing spontaneous apoptosis: Cytospins of neutrophils are made after overnight culture @37° C. Cells are stained using the Rapi Diff (II) system and examined by microscopy for morphological signs of apoptosis such as DNA condensation and blebbing.

Table 2: % neutrophils positive for superoxide anion (O₂⁻) production. Isolated neutrophils are cultured for the indicated time period. Fifteen minutes prior to acquisition cells are incubated with 10 μM DHE. The percentage of cells positive for superoxide production is determined by examining increased fluorescence (FL2) above background.

Table 3: Gene expression regulation associated with ‘Model Cell-System/Discovery Assay’ to study the molecular mechanisms of cellular response to ROS and apoptosis, in primary human neutrophils. Total cellular RNA isolated from human neutrophils undergoing spontaneous ROS-induced apoptosis is used as a probe for mRNA expression analysis using Incyte human ‘LifeGrid’ microarrays. Expression levels (normalised across the arrays) is presented for 0, 2, 3, 4, 5 and 6 hours post-isolation. NB. Hs.##### numbers correspond to NCBI ‘Unigene’ sequence database identifiers.

Table 4. Gene expression changes correlate across various models that share the induction of apoptosis by ROS. Shown here is the ‘early’ regulation of a) BCL2/adenovirus E1B 19 kD-interacting protein 3-like (Hs.132955) mRNA, and b) retinoic acid receptor responder (tazarotene induced) 3 (Hs.17466). Total cellular RNA isolated from human neutrophils and HeLa cells undergoing ROS-induced apoptosis is used as a probe for mRNA expression analysis using Incyte human ‘LifeGrid’ microarrays. Expression levels (normalised across the arrays) is presented for 0, 2, 3, 4, 5 and 6 or 24 hours post-treatment. NB. Hs.##### numbers correspond to NCBI ‘Unigene’ sequence database identifiers.

Table 5: Gene expression regulation associated with ‘Model Cell-System/Discovery Assay’ to study the molecular mechanisms of cellular response to ROS and apoptosis, in cisplatin-treated HeLa cells. Total cellular RNA isolated from HeLa cells undergoing endogenous ROS-induced apoptosis is used as a probe for mRNA expression analysis using Incyte human ‘LifeGrid’ microarrays. Expression levels (normalised across the arrays) is presented for 0, 2, and 4 hours post-treatment.

Table 6. Gene expression changes correlate across various models that share the induction of apoptosis by ROS. Shown here is the ‘early’ up-regulation of genes that correlate/cluster between both HeLa treated with cisplatin and HeLa treated with UV-irradiation Total cellular RNA isolated from HeLa cells undergoing ROS-induced apoptosis is used as a probe for mRNA expression analysis using Incyte human ‘LifeGrid’ microarrays. Expression levels (normalised across the arrays) is presented for 0, 2, and 4 h post-treatment. NB. Hs.##### numbers correspond to NCBI ‘Unigene’ sequence database identifiers.

Table 7: Gene expression regulation associated with ‘Model Cell-System/Discovery Assay’ to study the molecular mechanisms of cellular response to ROS and apoptosis, in UV-irradiated HeLa cells. Total cellular RNA isolated from HeLa cells undergoing endogenous ROS-induced apoptosis is used as a probe for mRNA expression analysis using Incyte human ‘LifeGrid’ microarrays. Expression levels (normalised across the arrays) is presented for 0, 2, 4 and 6 hours post-treatment.

Table 8: Gene expression regulation associated with ‘Model Cell-System/Discovery Assay’ to study the molecular mechanisms of cellular response to ROS and apoptosis, in H₂O₂ treated HL60 cells. Total cellular RNA isolated from HL60 cells and used as a probe for mRNA expression analysis using Incyte human ‘LifeGrid’ microarrays. Expression levels (normalised across the arrays) is presented for 0 and 2 hours post-isolation. NB. Hs.##### numbers correspond to NCBI ‘Unigene’ sequence database identifiers.

Table 9: Gene expression regulation associated with ‘Model Cell-System/Discovery Assay’ to study the molecular mechanisms of cellular response to ROS and apoptosis, in H₂O₂ treated HeLa cells. Total cellular RNA isolated from HeLa cells and used as a probe for mRNA expression analysis using Incyte human ‘LifeGrid’ microarrays. Expression levels (normalised across the arrays) is presented for 0 and 2 hours post-isolation. NB. Hs.##### numbers correspond to NCBI ‘Unigene’ sequence database identifiers.

### DETAILED DESCRIPTION OF THE INVENTION

The regulation of many genes presently known to be involved in apoptosis, including caspase genes, is presently being investigated as a route to the manipulation of apoptotic processes. Such attempts suffer from a fundamental flaw, in that because many of these genes/proteins become involved in apoptosis after the cell has made a commitment to the apoptotic pathway, regulation of these genes can only serve to delay the onset of apoptosis and not to prevent it. The present invention avoids this fundamental drawback and provides a method by which genes involved in the early stages of apoptosis, whose expression is modulated by reactive oxygen species in the cell, can be identified and isolated.

It is known that a change in intracellular REDOX or an increase in ROS levels triggers apoptosis, but the mechanisms by which this operates have been unknown. According to the present invention, these mechanisms involve ROS-responsive modulation of signal transduction cascades, and/or changes in protein and/or mRNA levels, which may be measured by the methods provided by the invention. An understanding of these mechanisms will provide the means for the development of therapeutics to modulate and/or control apoptotic processes (Hetts S W, JAMA Vol 279(4) p300-307).

For example, the control of apoptosis in neutrophils is useful in the treatment of a number of diseases, including asthma, COPD, posttraumatic acute respiratory distress syndrome (ARDS), systemic lupus erythematosus (SLE), Inflammatory Bowel disease (IBD), end-stage renal disease (uremia), Cardiopulmonary bypass, rheumatoid arthritis, cutaneous allergic (leukocytoclastic) vasculitis (CAV), cystic fibrosis (CF), severe congenital neutropenia (SCN), Endotoxin (ET)-induced liver failure, acute myelogenous leukaemia and neutropenia following radiation and chemotherapeutic treatments for cancer.

Many chemotherapeutic agents used in the treatment of cancer kill the tumour cells by the induction of apoptosis mediated through the generation of ROS. A problem with this approach is that many tumour cells, through elevated metabolism, have increased resistance to ROS-mediated apoptosis; higher levels of chemotherapeutic agents that have to be used to overcome this resistance are associated with increased toxicity and side effect.

The present invention addresses this fundamental drawback and provides a method by which genes involved in the cells protective response mechanisms to ROS, and whose expression is modulated by reactive oxygen species in the cell, can be identified and isolated. An understanding of these mechanisms will provide the means for the development of therapeutics to modulate a cancer cells ability to resist chemotherapeutic drugs and thus allow a reduction in the levels of chemotherapeutic drug administered.

### GENERAL TECHNIQUES

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference), chemical methods, pharmaceutical formulations and delivery and treatment of patients.

### REACTIVE OXYGEN SPECIES

The production of ROS may be induced in cells by the partial reduction of oxygen. Accordingly, administration of agents to cells which interfere with respiratory mechanisms is effective in the generation of ROS. For example buthionine sulphoximine (BSO) which interferes with the synthesis of glutathione lowers the anti-oxidant defences in the cell and as a result renders the cell more susceptible to ROS mediated apoptosis (Fernandes and Cotter, Biochem Pharmacol. 1994 17:675-81). 2-Methoxyoestradiol an inhibitor of superoxide dismutase acts in a related manner by blocking the ability of its target enzyme to dismutate superoxide radicals generated by either normal metabolic processes or other external ROS generating agents.

Externally applied agents which induce ROS production in cells include UV irradiation, disruptors of the mitochondrial respiratory chain, cisplatin and various alkaylating agents. For example, bleomycin treatment of tumour cells leads to the production of ROS and this in turn drives apoptosis (Proc Natl Acad Sci U S A 1998 95:5061-6). N-(4-Hydroxyphenyl) retinamide (4HPR) is currently used in cancer prevention and therapy trials. It is thought that its effects result from induction of apoptosis. 4HPR-induced apoptosis in human cervical carcinoma C33A cells involves enhanced generation of reactive oxygen species (ROS) (Oncogene 1999 November 4; 18(46):6380-7).

Certain cell types, such as primary neutrophils, undergo spontaneous apoptosis on cloturing. This apoptosis is accompanied by induction of genes associated with ROS pathways, and provides a good model of ROS-associated apoptosis in primary cells. Advatageously, the primary netrophils are cultured in a medium containing serum, which provides some stabilisation of the spontaneous apoptosis.

ROS may moreover be administered to cells in the form of exogenously added ROS. Typically, this may be in the form of H₂O₂.

### DETERMINATION OF EXPRESSION LEVELS

A number of individual gene product types may be screened for in the present invention. These products include polypeptides and nucleic acids (N.B. actual examples are only given for nucleic acids, but may also be considered to include polypeptides). The expression levels assessed may be absolute levels of production of a particular polypeptide or nucleic acid, or the levels of production of a derivative of any polypeptide or nucleic acid. For example, the invention may be configured to measure the level of expression of a particular mRNA splice variant, or the amount present of a phosphorylated derivative of a particular polypeptide.

Where it is desired to monitor the levels of expression of a known gene product, conventional assay techniques may be employed, including nucleic acid hybridisation studies and activity-based protein assays. Kits for the quantitation of nucleic acids and polypeptides are available commercially.

Where the gene product to be monitored is unknown, however, methods are employed which facilitate the identification of the gene product whose expression is to be measured. For example, where the gene product is a nucleic acid, arrays of oligonucleotide probes may be used as a basis for screening populations of mRNA derived from cells.

### ARRAYS

Gene Arrays of oligonucleotides specific to gene sequences archived in public domain databases, such as GenBank, are available commercially from a number of suppliers (such as Incyte Genomics, USA). Examples of such commercial arrays are in the form of either nucleotides spotted onto a membrane filter (such as nitrocellulose), or a solid support (such as glass). Commercial Gene Arrays are used to profile the patters of gene expression which are associated with the process of apoptosis in neutrophils, and other cell types.

Gene Arrays are additionally constructed in-house, by spotting nucleotide sequences derived from cDNA clones generated from in-house libraries or from cDNA clones purchased commercially. Such arrays allow the expression profiling of proprietary and/or novel nucleotide sequences.

Gene Arrays are additionally constructed by commercial sources (e.g. Genescreen), by spotting nucleotide sequences derived from cDNA clones generated from in-house libraries or from cDNA clones purchased commercially. Such arrays allow the expression profiling of proprietary and/or novel nucleotide sequences.

Many of the cDNA sequences or EST (expressed sequence tag) sequences deposited in the public domain databases are derived from a restricted set of tissue types, such as liver, brain and foetal tissue. The cloning of in-house cDNA libraries which are focused to specific cellular events, such as ROS-mediated apoptosis offers the possibility to identify, clone and characterise novel genes which are associated with this process. Similarly, the cloning of in-house cDNA libraries which are focused to specific tissue types, such as the neutrophil, offers the possibility to identify, clone and characterise novel genes whose expression is restricted to this cell type. Libraries (cDNA) constructed using a physical subtraction, such as the ClonTech ‘Select’ SSH method (suppression hybridisation) and novel modifications of such, as described, allow the selective cloning of genes whose expression is differentially regulated in the process or cell type being studied. Gene Array technology is combined with SSH cDNA libraries to identify false-positives and further focus on truly differentially expressed genes. Clones from each SSH library constructed are picked, cultured and archived as glycerol stocks. The cDNA inserts contained within individual plasmid clone are PCR amplified and spotted onto in-house arrays. Differential expression is confirmed using hybridisation with a radiolabelled probe generated from the mRNA used for each reciprocal subtractions.

Arrays of nucleic acids may be prepared by direct chemical synthesis of nucleic acid molecules. Chemical synthesis involves the synthesis of arrays of nucleic acids on a surface in a manner that places each distinct nucleic acid (e.g., unique nucleic acid sequence) at a discrete, predefined location in the array. The identity of each nucleic acid is determined by its spatial location in the array. These methods may be adapted from those described in U.S. Pat. No. 5,143,854; WO90/15070 and WO92/10092; Fodor et al. (1991) Science, 251: 767; Dower and Fodor (1991) Ann. Rep. Med. Chem., 26: 271.

In a preferred aspect of the invention, arrays of nucleic acids may be prepared by gridding of nucleic acid molecules. Oligonucleotides may be advantageously arrayed by robotic picking, since robotic techniques allow the most precise and condensed gridding of nucleic acid molecules; however, any technique, including manual techniques, which is suitable for locating molecules at discrete locations on a support, may be used.

The gridding may be regular, such that each colony is at a given distance from the next, or random. If molecules are spaced randomly, their density can be adjusted to statistically reduce or eliminate the probability of overlapping on the chosen support.

Apparatus for producing nucleic acid microarrays is available commercially, for example from Genetix and Genetic Microsystems. Moreover, pre-prepared arrays of nucleic acid molecules are available commercially, for example from Incyte Genomics Inc. (Human LifeGrid⁽™⁾). Such arrays will comprise expressed sequence tags (ESTs) representative of most or all the genes expressed in a cell or organism, thus providing a platform for the screening of mRNA populations from multiple ROS-treated cells.

Samples for mRNA population analysis may be isolated and purified by any suitable mRNA production method; for example an RNA isolation kit is available from Stratagene.

### GENERAL APPLICATIONS OF THE INVENTION

The genomics strategy, and model systems or discovery assays described herein provides the means to characterise the molecular mechanisms of apoptosis. In particular, these molecular mechanisms include the initiation of apoptosis induced by ROS, the molecular signalling pathways mediating or associated with apoptosis induced by ROS, and the molecular mechanisms mediating or associated with the cell defence response to ROS. In addition, the strategy provides the means to identify, characterise, and clone molecules, including oligonucleotides and polypeptides, associated, both causally and consequentially, to apoptosis.

In a preferred configuration of the present invention, cells such as primary human neutrophils are isolated and purified from the peripheral blood of individuals. Upon culture in a serum-containing cell culture medium these neutrophils undergo ‘spontaneous apoptosis’ (Haslett, Clinical Science 83, pp639-648, 1992). Apoptosis as measured by morphology is apparent and increases from approximately 6 to 8 hours following isolation of the cells. The onset of apoptosis is further characterised using additional markers for apoptosis, such as caspase activation and cell shrinkage. These assays serve to identify the earliest measurable onset of the cells commitment to apoptosis. Intracellular events which drive this commitment of the cell to apoptosis occur before and around this earliest measurement of the commitment.

An intracellular initiator of the commitment to apoptosis is a change in REDOX manifested by an increase in level of ROS (such as O₂⁻ or H₂O₂). Intracellular ROS is measured to confirm this increase preceding the measurement of apoptosis. Indeed this is substantiated by the finding that a burst of O₂⁻ is detected prior to the detection of apoptosis, and which is first detected from approximately 6 hours, peeks at 3 to 4 hours and has decreased to base-line levels by 5 hours following isolation of the cells.

Suitable inhibitors of ROS include antioxidants such as NAC, or inhibitors of NADPH oxidase (which has been shown to generate O₂ release in neutrophils) e.g. DPI, are used to confirm the causal role of ROS in mediating apoptosis in this model system.

The characterisation of the apoptosis process and ROS involvement in this model system serve to establish a temporal window between the onset of ROS (which is an initial trigger for the apoptosis process) and the measurement of cellular apoptosis (at which point the cell is committed to undergoing apoptosis). The key early intracellular events which control the initiation of apoptosis induced by ROS, the molecular signalling pathways mediating or associated with apoptosis induced by ROS, and the molecular mechanisms mediating or associated with the cell defence response to ROS (and those which may represent the most attractive therapeutic targets) occur within this window.

Global gene expression patterns allows a detailed characterisation of the mechanisms underlying and causal to the apoptotic process. Indeed we show that ‘known’ apoptosis genes are regulated at 2 to 4 hours. We also confirm the role for ROS in neutrophil spontaneous apoptosis by the demonstration that key ‘known’ mediators of the cells response to REDOX change and oxidative (ROS) stress, are regulated at 2 to 4 hours.

Analysis of global gene expression patterns, using for example ‘cluster analysis’ to group genes with similar temporal patterns of expression, allows a detailed characterisation of signalling pathways and cellular processes associated with treatments. Indeed we show that this analysis, of gene expression using our model system, identifies genes that are ‘known’ to be co-regulated and co-expressed, and which belong to the same signalling pathways or cellular process. We also show that many genes (the expression patterns of which are grouped, or clustered, with those genes that have know function) identified and cloned have little or no know known biological function, or which have no existing sequence homology to previously cloned genes. These genes may represent the most attractive therapeutic targets.

GM-CSF is a well-characterised neutrophil growth/survival factor. Culture of human neutrophils in the presence of GM-CSF inhibits apoptosis and prolongs survival for up to 24 to 48 hours (Brach et al., Blood 80:2920-2924). The culture of neutrophils in GM-CSF provides a very useful additional model system with which to study neutrophil apoptosis. Indeed we show, from analysis of global gene expression patterns, that GM-CSF regulates the ‘early’ expression of many of the ‘known’ genes that are part of the cells defence mechanism to ROS-mediated oxidative stress; specifically that induced by O₂⁻ or H₂O₂ . We also show that many genes (the expression patterns of which are grouped, or clustered, with those genes that have know function) identified and cloned have little or no know known biological function, or which have no existing sequence homology to previously cloned genes. These genes may represent the most attractive therapeutic targets.

In a further preferred configuration of the present invention, cells such as HL60 or HeLa, are cultured and treated with external agents that induce apoptosis through the endogenous production of ROS, such as UV-irradiation, cytokines such as TNFα, or chemotherapeutic agents such as cisplatin. Upon treatment of cells with UV-irradiation, TNFα, or cisplatin, apoptosis is induced; this apoptosis has been shown to be mediated by an endogenous production of ROS (Miyajima A, Nakashima J, Yoshioka K, Tachibana M, Tazaki H, Murai M (1997) Role of reactive oxygen species in cis-dichlorodiammineplatinum-induced cytotoxicity on bladder cancer cells. Br J Cancer 76 (2) pp206-10; Chan, W-H, Yu, J-S (2000) Inhibition of UV Irradiation-induced oxidative stress and apoptotic biochemical changes in human epidermal carcinoma A431 cells by genestein. J. Cell Biochem. 78 pp73-84; Sidoti-de Fraisse C, Rincheval V, Risler Y, Mignotte B, Vayssiere J L (1998) TNF-alpha activates at least two apoptotic signalling cascades. Oncogene (13): pp1639-51). Apoptosis as measured by morphology is apparent as determined by DNA fragmentation. The onset of apoptosis is characterised using markers for apoptosis. These assays serve to identify the earliest measurable onset of the cells commitment to apoptosis. Intracellular events which drive this commitment of the cell to apoptosis occur before and around this earliest measurement of the commitment.

As described, global gene expression patterns allows a detailed characterisation of the mechanisms underlying and causal to the apoptotic process. Indeed we show that ‘known’ apoptosis genes are regulated ‘early’ at 2 to 4 hours. We also confirm the role for ROS in apoptosis by the demonstration that key ‘known’ mediators of the cells response to REDOX change and oxidative (ROS) stress, are regulated at 2 to 4 hours.

Analysis of global gene expression patterns, using for example ‘cluster analysis’ to group genes with similar temporal patterns of expression, allows a detailed characterisation of signalling pathways and cellular processes associated with treatments. Indeed we show that this analysis, of gene expression using our model system, identifies genes that are ‘known’ to be co-regulated and co-expressed, and which belong to the same signalling pathways or cellular process. We also show that many genes (the expression patterns of which are grouped, or clustered, with those genes that have know function) identified and cloned have little or no know known biological function, or which have no existing sequence homology to previously cloned genes. These genes may represent the most attractive therapeutic targets.

In a further preferred configuration of the present invention, cells such as HL60 or HeLa, are cultured and treated with exogenous ROS, such as H₂O₂ . Upon treatment of cells with H₂O₂ apoptosis is induced, as measured by DNA fragmentation. The onset of apoptosis is characterised using markers for apoptosis. These assays serve to identify the earliest measurable onset of the cells commitment to apoptosis. Intracellular events which drive this commitment of the cell to apoptosis occur before and around this earliest measurement of the commitment.

Signal Transduction events are associated with induction of commitment of cells to undergoing apoptosis. Signal transduction would typically involve events such as protein phosphorylation and/or de-phosphorylation.

The invention provides the means to allow the identification and isolation of genes or polypeptides that may represent valuable and attractive therapeutic targets for the control of apoptosis in a range of conditions and diseases.

### EXAMPLE 1

Establishment and Characterisation of a ‘Model Cell-System/Discovery Assay’ to Study the Molecular Mechanisms of Cellular Response to ROS and Apoptosis, in Primary Human Neutrophils.

This example describes the establishment of a model cell system in primary human neutrophils. The isolation and culture of primary human neutrophils is described. Neutrophils are allowed to undergo spontaneous apoptosis in culture. This spontaneous apoptosis is accompanied and characterised by the endogenous production of ROS. This ‘Model Cell System’ forms the basis of a novel ‘Discovery Assay’ that is then used for the characterisation, identification and functional validation of nucleotide (genes, mRNAs) or polypeptide (proteins, peptides) sequences associated with and potentially responsible for the molecular mechanisms of cellular response to ROS and apoptosis, including; 1) the initiation of the apoptosis process, 2) the cellular signalling pathways, and 3) the cells attempt to combat ROS and apoptosis in the cell.

We demonstrate the utility of this model system to ‘discover’ early regulated genes associated with ROS-induced apoptosis, by the discovery of genes that have ‘known’ involvement in cell apoptosis, carcinogenesis, signal transduction and the cellular response to ROS, oxidative and environmental stress. In many cases these examples include genes, identified by cluster analysis within and between different ROS-induced apoptosis models, that share coordinate regulation and which belong to the same signal transduction pathways or cellular processes. Similarly we demonstrate the identification of genes regulated ‘early’ in ROS-induced apoptosis that have no known biological function i.e. ESTs from both commercial filters and from our own libraries of differentially expressed genes isolated by Suppression Subtractive Hybridisation (SSH). By association with known genes, as described, these novel genes themselves are identified as candidate apoptosis genes that may similarly function in 1) the initiation of the apoptosis process, 2) the cellular signalling pathways, and 3) the cells attempt to combat ROS and apoptosis in the cell.

Isolation and Culture of Primary Human Neutrophils

Whole blood (20-50 ml) is taken from normal healthy volunteers by venepuncture. Coagulation is prevented by the use of sodium citrate. A 6% dextran (mol wt 509,000; Sigma) saline solution is added in 1:4 ratio to whole blood and the erythrocytes allowed to sediment for 45 minutes at 22° C. The buffy coat is then under-layered with 5 ml Ficoll-Paque (Pharmacia LKB Biotechnology) and centrifuged (300 g, 30 min) to pellet granulocytes and erythrocytes (Boyum, 1968). The pellet is resuspended in 1 ml cell culture tested water (Sigma) for 40 sec., followed by the addition of 14 ml Hanks buffer (Sigma) and centrifuged (300 g, 10 min.). This lysis step is repeated to ensure removal of all erythrocytes. The remaining pellet is resuspended in RPMI 1640 supplemented with 10% foetal calf serum (Sigma), L-glutamine (2 mM), penicillin (100 U/mI; Sigma), streptomycin (100 μg/ml; Sigma) and amphotericin B (2.5 μg/ml; Sigma). Cell number and viability is checked using trypan blue exclusion (Boyum, (1968) Scand J Clin Lab Invest Suppl; 97:77-89).

Isolated neutrophils are maintained at a density of 2×10⁶/ml in RPMI 1640 supplemented with 10% foetal calf serum (Sigma). Further additions to the medium included L-glutamine (2 mM), penicillin (100 U/ml), streptomycin (100 μg/ml) and amphotericin B (2.5 μg/ml) (Sigma). Cells are incubated at 37° C. in a humidified CO₂ (5%) incubator.

Cellular/Biochemical Characterisation of Apoptosis

A range of assays are established and used to measure the magnitude and temporal induction of apoptosis. The earliest biochemical measurement of the apoptosis phenotype by these assays is considered to be the point beyond which the cells are ‘Committed’ to the process of apoptosis. In addition, these measurements determine the reproducibility of induction of apoptosis in the model systems. Furthermore, these measurements determine the cellular mechanisms of apoptosis in these systems (such as whether apoptosis is caspase-dependent or caspase-independent).

Primary human neutrophils are isolated and purified from the peripheral blood of normal healthy individuals. Upon culture in a serum-containing cell culture medium these neutrophils undergo ‘spontaneous apoptosis’ (Haslett, Clinical Science 83, pp639-648, 1992).

Our data indicate the earliest detection of neutrophil spontaneous apoptosis, and thus a time of ‘commitment to die’ at 6 to 8 hours post-isolation.

Caspase Activation Assay

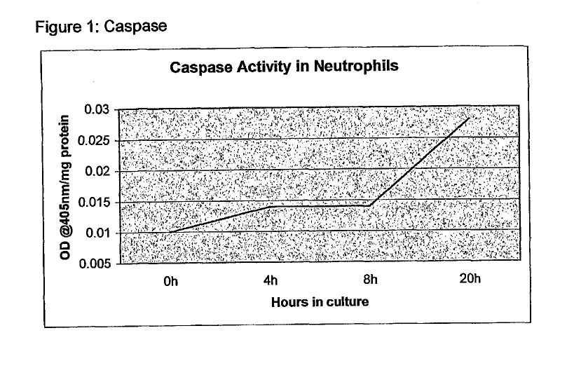

Caspase activity (Caspase-3) is measured using a commercial kit (CaspACE™ Assay System, Promega). The methodology is essentially as described by the manufacturer. Cells are removed from culture and centrifuged (300 g/10 min) at 4° C. The pellet is kept on ice, washed in ice-cold Hanks buffer and then resuspended in Cell Lysis Buffer at 10⁸/ml. Cells are lysed by freeze-thaw once, incubated on ice for 15 min, followed by centrifugation (15,000 g/20 min) at 4° C. The caspase 3 activity present in the supernatant fraction is measured using the absorbance at 405 nm and normalised per mg of protein in the supernatant. FIG. 1 represents the increase in caspase activity during the incubation period. While caspase activity is observed at the earlier time points, there is a dramatic increase between 8 and 20 h of culture, which is in agreement with our morphological data.

Morphological Determination of Apoptosis

A cell aliquot (100 μl) is removed from culture and using an IEC Centra-7 centrifuge equipped with a Cytobucket™ adapter, a monolayer of cells is concentrated onto standard microscopic slides. Preparations are allowed to air dry prior to fixing in Rapi-Diff (Diagnostic Developments, UK) solution A (reactive ingredient 100% methanol). Slides are air dried prior to immersion in solution B containing; eosin Y (0.1% w/v), formaldehyde (0.1% w/v), sodium phosphate dibasic (0.4% w/v) and potassium phosphate monobasic (0.5% w/v). Excess stain is drained from the slide prior to immersion in solution C, containing methylene blue (0.4% w/v), Azure A (0.04% w/v), sodium phosphate dibasic (0.4% w/v), potassium phosphate monobasic (0.05% w/v) and potassium phosphate monobasic (0.4% w/v), to counterstain the cytoplasm. Excess dye is rinsed; the slides air-dried and mounted in DPX aqueous mountant (BDH Laboratory Supplies, U.K.). Morphological examination is then carried out by light microscopy for the presence of apoptotic cells as determined by the loss of membrane asymmetry and condensation of cytoplasm and nuclei (Cotter and Martin, 1996).

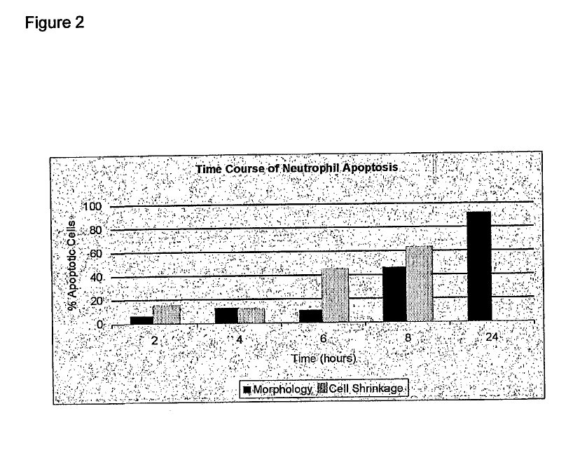

Apoptosis as measured by morphology is apparent and increased from approximately 6 hours following isolation of the cells. Our results demonstrate that although there is a small amount of apoptosis occurring at 2 h post isolation, there is a large increase in the number of cells undergoing spontaneous apoptosis between 6-8 h resulting in close to 50% of the cells showing apoptotic morphology at 8 hours (See Table 1 and FIG. 2).

Cell Shrinkage Determination of Apoptosis

Onset of apoptosis as determined by cell shrinkage was determined by flow cytometry. Isolated cells are set up in culture at a concentration of 2×10⁶/ml. At the indicated time points a sample is removed and the forward and side scatter parameters of the cells are measured by flow cytometry using a Becton Dickenson FACScan equipped with CellQuest software. Cell shrinkage is detected by a reduction in forward scatter parameters. Determination of apoptosis inhibition was calculated by the percentage of cells with reduced forward scatter parameters, in untreated cultures, minus percentage of cells with reduced forward scatter parameters in treated cultures.

Our results for spontaneous neutrophil apoptosis demonstrate that although there is a small amount of apoptosis occurring at 2 h and 4 h post isolation, there is a large increase in the number of cells undergoing spontaneous apoptosis from 6 h with close to 50% of the cells showing apoptotic morphology at 6 hours (See FIG. 2).

Neutrophil Spontaneous Apoptosis is Accompanied by the Endogenous Production of ROS, in the Form of O₂⁻ and H₂O₂

Human neutrophils are specialised for the phagocytosis and lysis of bacteria. This lysis is accompanied by an enzyme complex called NADPH oxidase (Babior, B. M. (1978)). Oxygen dependent microbial killing by phagocytes. New England Journal of Medicine, 298(12) pp659-668. NADPH oxidase is assembled at the membrane of the phagosome and generates ROS, in the form of superoxide (O₂⁻). This endogenous production of ROS contributes to the bacterial lysis. NADPH oxidase activity is present in normal peripheral blood neutrophils and is further increased by cellular activation. Consequently, neutrophils have the capacity to generate significant amounts of endogenous ROS. As with other cell types, ROS in the form of superoxide may also be produced by mitochondria during oxidative respiration.

Chronic Granulomatous Disease (CGD) is characterised by a molecular defect and loss of activity of the neutrophil NADPH Oxidase. Notably, neutrophils isolated from patients with CGD have a significantly delayed spontaneous apoptosis Kasahara Y, lwai K, Yachie A., Ohta, K., Konno A., Seki H., Miyawaki, T and Taniguchi N. (1997) Involvement of reactive oxygen intermediates in spontaneous and CD95(Fas/Apo-1)-mediated apoptosis of neutrophils. Blood 89 (5) pp1748-1753.

Increased H₂O₂ occurs upon neutrophil activation, with. e.g. PMA, and this is associated with increased rate of apoptosis (Lundqvist-Gustafsson H, and Bengtsson T. (1999) Activation of the granule pool of the NADPH oxidase accelerates apoptosis in human neutrophils. J. Leuk. Biol. 65: pp196- 204).

Our results demonstrate that we can detect an increase in the superoxide production, as detected by flow cytometry (See Table 2). The greatest number of cells producing superoxide anions peaked 3-4 hours post isolation, before returning to background levels.

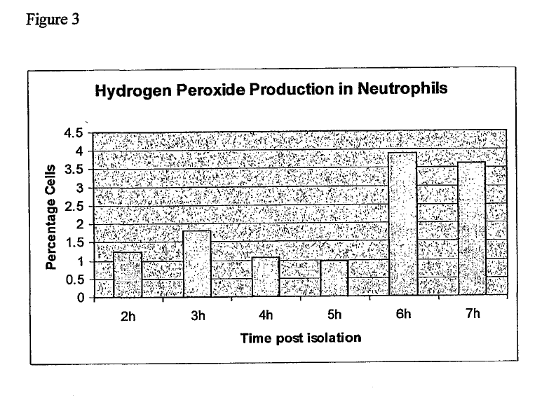

Our results also demonstrate that this peak in O₂⁻ production precedes an increase in intracellular H₂O₂ production (See FIG. 3). This temporal pattern is consistent with the H₂O₂ production resulting from the action of superoxide dismutase, an enzyme present in neutrophils and which catalyses the conversion of O₂⁻ to H₂O₂

Inhibitors of Neutrophil NADPH Oxidase Inhibit Neutrophil Spontaneous Apoptosis

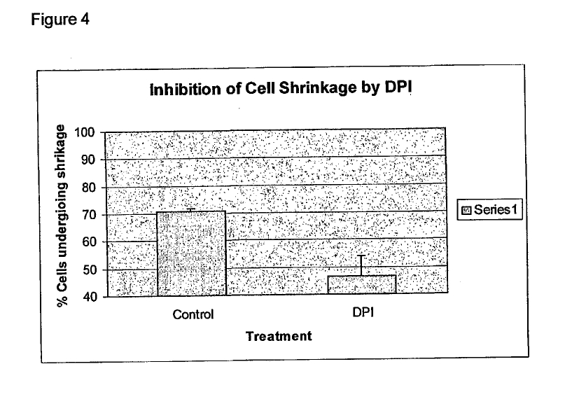

Primary human neutrophils are isolated and puriied from the peripheral blood of normal healthy individuals. Upon culture for 6 hour in a serum-containing cell culture medium, these neutrophils undergo ‘spontaneous apoptosis’ (Haslett, Clinical Science 83, pp639-648, 1992). As cell shrinkage is one of the main characteristics of cells undergoing apoptosis (Wesselbory et al., Cell Immunol. 148, 234-41, 1993), the process of apoptosis can be followed by examining forward scatter parameters of neutrophils using flow cytometry. FIG. 4 demonstrates the effect of the NADPH inhibitor DPI on neutrophil apoptosis as determined by cell shrinkage. From the figure it is clear that by inhibiting NADPH oxidase by DPI, one can decrease the percentage of cells undergoing apoptosis.

Neutrophil Spontaneous Apoptosis is Associated With Significant Changes in Gene Expression.

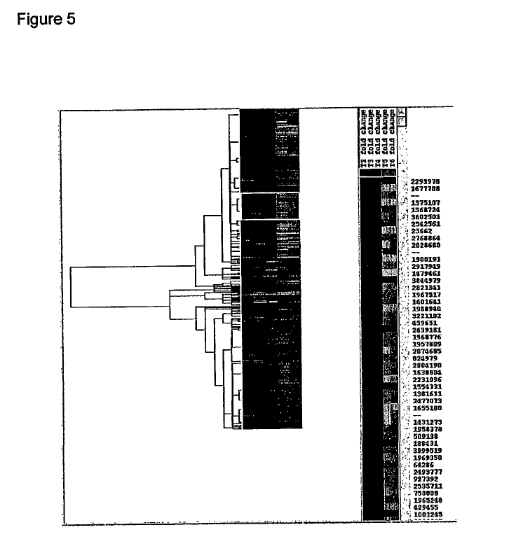

Primary human neutrophils are isolated and purified from the peripheral blood of normal healthy individuals. At various time points post-isolation, total cellular RNA is isolated and examined for gene expression changes using microarray. Significant transcription of mRNA is observed. In one example experiment, as many as 2500 genes are detected using Incyte human ‘LifeGrid’ filters; these genes are increased or decreased by greater than 2-fold over a 6-hour time course as compared with the time zero control neutrophils. As many as 360 genes are increased or decreased as early as 2 hours post-isolation, and prior to the ‘commitment to die’ point identified as described. As many as 1800 genes are increased or decreased by 5 h post-isolation. FIG. 5 shows cluster analysis of genes regulated during neutrophil spontaneous apoptosis time course. Genes fall into primary clusters of up (red) and down-regulated (green) genes. By 5 hours post-isolation ˜1200 genes are up-regulated and ˜600 down-regulated.

Many of these regulated genes are ESTs (˜400) with no known biological functional annotation. Many more (data not shown) have only very little biological characterisation.

This significant gene expression regulation associated with spontaneous apoptosis, is on a background of 1) a low baseline of gene expression compared to other cell types (data not shown), and 2) the absence of many of the genes commonly associated with cellular mitosis, since the neutrophil is a non-dividing cell. These factors contribute to making this an ideal model for the study and identification of genes associated with cellular processes such as apoptosis.

Changes in ‘Early’ Gene Expression Include Classes of Genes With ‘Known’ Roles in Apoptosis.

Of those genes regulated as early as 2 hours post-isolation, classical apoptosis-related genes including both caspase and BcI-2 related genes are detected (See Table 3). Caspase 10, is particularly enriched in haematopoietic cells and the chronic myelogeous leukaemia cell line K562. BCL2-associated athanogene 5 (BAG5) modulate and antagonise the chaperone functions of heat shock proteins (e.g. HSP-70). Heat shock proteins stabilise protein structure and function during cellular stress. BCL2/adenovirus E1B 19 kD-interacting protein 3-like mRNA (BNIP3L) is also regulated. Overexpression of BNIP3L has been shown to be pro-apoptotic, and can overcome the antiapoptotic effects of BCL2 and BCL2L1 in transfected cells (Chen, G.; Cizeau, J.; Vande Velde, C.; Park, J. H.; Bozek, G.; Bolton, J.; Shi, L.; Dubik, D.; Greenberg, A.: Nix and nip3 form a subfamily of pro-apoptotic mitochondrial proteins. J. Biol. Chem. 274: 7-10, 1999. and Matsushima, M.; Fujiwara, T.; Takahashi, E.; Minaguchi, T.; Eguchi, Y.; Tsujimoto, Y.; Suzumori, K.; Nakamura, Y. : Isolation, mapping, and functional analysis of a novel human cDNA (BNIP3L) encoding a protein homologous to human NIP3. Genes Chromosomes Cancer 21: 230-235, 1998.). These ‘early regulated’ genes represent potential regulators of the ROS-induced neutrophil spontaneous apoptosis.

Changes in ‘Early’ Gene Expression Include Cell-Cycle Regulator Genes With ‘Known’ Involvement in Apoptosis.

Both cyclin-dependent kinase 2 (CDK2) and p21 cyclin-dependent kinase inhibitor 1a (CDKN1A) are regulated early post-isolation (See Table 3). The expression, in neutrophils, of cell-cycle regulating genes is particularly interesting since neutrophils are a terminally differentiated cell type which do not replicate. Apoptosis of human endothelial cells after growth factor deprivation is associated with a rapid increase in CDK2 activity. CDK2 remains inactive in the presence of the nuclear inhibitor CDKN1A. Upon cleavage by caspase activation, CDKN1A releases is block on CDK2 activity and exits the nucleus. These ‘early regulated’ genes represent potential regulators of the ROS-induced neutrophil spontaneous apoptosis.

Changes in ‘early’ gene expression include genes associated with an oncogenic phenotype or disease, or associated with apoptosis.

A variety of genes associated with an oncogenic phenotype or disease, or associated with apoptosis are also regulated ‘early’ post-isolation of neutrophils in this model (See Table,3). These genes include Death-associated protein (DAP), a homologue of DAP; serine/threonine protein kinase 17B and programmed cell death 5. These ‘early regulated’ genes represent potential regulators of the ROS-induced neutrophil spontaneous apoptosis.

Changes in Gene Expression Include Genes Associated Retinoids and Cellular Differentiation.

Neutrophil differentiation from myeloid progenitor cells may be facilitated by treatment with Vitamin A/Retinoic acid. Indeed certain myeloid leukaemias are treated with retinoic acid, which promotes neutrophil differentiation (Clin Lab Sci 2000 Spring; 13(2):98-105; Acute promyelocytic leukaemia (AML-M3)-Part 1: Pathophysiology, clinical diagnosis, and differentiation therapy. Randolph TR). HL60 cells used here are an acute promyelocytic leukaemia, associated with the 15; 17 translocation; retinoic acid binding protein CRABP1 is transposed in this form. HL60 cells are also differentiated by retinoic acid, which removes the proliferative block. Spontaneous neutrophil apoptosis is associated with the early regulation of CRABP1, retinoic acid receptor beta (RARB); which has been associated with Parkinson disease, a disease that is characterised by apoptosis of a sub-set of neuronal cells (Kreczel, W.; Ghyselinck, N.; Samad, T. A.; Dupe, V.; Kastner, P.; Borrelli, E.; Chambon, P.: Impaired locomotion and dopamine signalling in retinoid receptor mutant mice. Science 279: 863-867, 1998.) and retinoic acid receptor responder 3 (RARRES3). RARRES3 is induced by Tazarotene, a RARB-selective retinoid used clinically for the treatment of psoriasis, a hyperproliferative and inflammatory skin disease. Tazarotene induces keratinocye differentiation/apoptosis. Tazarotene also induced RARRES3 expression in retinoid-sensitive but not retinoid-resistant breast cancer cells. Overexpression of RARRES3 inhibited the growth of many cell lines (DiSepio, D.; Ghosn, C.; Eckert, R. L.; Deucher, A.; Robinson, N.; Duvic, M.; Chandraratna, R. A. S.; Nagpal, S.: Identification and characterisation of a retinoid-induced class II tumour suppressor/growth regulatory gene. Proc. Nat Acad. Sci. 95: 14811-14815, 1998.). These ‘early regulated’ genes, and genes associated with retinoid action represent potential regulators of the ROS-induced neutrophil spontaneous apoptosis.

Another gene, runt-related transcription factor AML1, is rapidly and transiently regulated ‘early’ post-isolation. Both retinoic acid RAR and AML1 transcription factors are found in leukaemias as fusion proteins. Association of these fusion proteins with histone deacetylase complex is required to block haematopoietic differentiation. We also show ‘early’ differential regulation of histone deacetylase 2,3, 6 and 7 (See Table 3).

Changes in ‘Early’ Gene Expression Include Genes Associated Cellular Response to ROS and Oxidative/Environmental Stress.

The following observations support and confirm a key ‘early’ regulatory role for ROS and oxidative stress in neutrophil spontaneous apoptosis, and in the regulation of ROS-related gene expression. Two ‘known’ key transcription regulators of the cellular response to oxidative stress are hypoxia-inducible factor 1 (HIF-1) and arylhydrocarbon receptor (AHR) (Morel, Y and Barouki, R. 1999. Repression of gene expression by oxidative stress. Biochem J. 342:481-496). HIF1 has a key role in cellular response to hypoxia, including regulating the genes involved in energy metabolism, angiogenesis and apoptosis. Both HIF1-alpha subunit and AHR are regulated ‘early’ in neutrophil spontaneous apoptosis (See Table 3). Of note is that HIF1-alpha has been shown to activate expression of BNIP3 (the homologue of which we describe here, regulated during spontaneous apoptosis), which in turn primes cells for apoptosis under conditions of persistent oxygen deprivation (Bruick, R. K.: Expression of the gene encoding the proapoptotic Nip3 protein is induced by hypoxia. Proc. Nat Acad. Sci. 97:9082-9087, 2000).

A recent publication, since the initial submission of this patent, has suggested that the action of AHR and the AHR transcribed genes represents a pivotal ‘upstream event’ in the apoptosis cascade, providing an intricate balance between promoting and preventing ROS-mediated oxidative stress (Biochem Pharmacol 2000 January 1;59(1):65-85, Role of the aromatic hydrocarbon receptor and [Ah] gene battery in the oxidative stress response, cell cycle control, and apoptosis. Nebert D W, Roe A L, Dieter M Z, Solis W A, Yang Y, Dalton T P).

The AHR is known to mediate some of its effects by increasing the expression of aldehyde dehydrogenase and quinone reductase, among other drug metabolising enzymes. We show here that four forms of aldehyde dehydrogenase are regulated during spontaneous neutrophil apoptosis as is a quinone oxidoreductase homologue.

Conditional disruption of the AHR nuclear translocator, in mouse, has recently suggested that this gene is responsible for the coordinated expression of both the AHR and HIF1 mRNA. This is consistent with our observations that the AHR and HIF1 mRNA are co-ordinately regulated in neutrophil spontaneous apoptosis (See Table 3).

NAD(P)H:quinone oxidoreductase (diaporase) has been reported as a part of the activation of defence mechanisms within the cell on exposure to xenobiotics, drugs and carcinogens (Benson, A. M.; Hunkeler, M. J.; Talalay, P.: Increase of NAD(P)H:quinone reductase by dietary antioxidants: possible role in protection against carcinogenesis and toxicity. Proc. Nat. Acad. Sci. 77: 5216-5220, 1980). Quinones are highly abundant in nature; their partial reduction can lead to redox cycling in the presence of molecular oxygen leading to the formation of highly reactive oxygen species. It has been shown to be induced by ROS in the form of exogenous H202 (Li, Y and Jaiswal, A K. 1994. Eur J Biochem. 226:31-39). We show here that diaphorase 4 (DIA4), is regulated ‘early’ post-isolation of neutrophils associated with spontaneous apoptosis (See Table 3). Interestingly, NAD(P)H:quinone oxidoreductase has been shown to be increased in chick embryos in response to the AHR ligand 2,3,7,8-tetrachlorodibenzo-p-dioxin, suggesting its regulation by this transcription factor (Spencer C B and Rifkind A B. 1990. Biochem Pharmacol 39:327-35), which is also found here, as described, to be regulated during spontaneous neutrophil apoptosis (See Table 3).

Another enzyme class, UDP-glucuronosyl transferases, associated with cell defence to xenobiotics and drugs, is also shown here to be regulated ‘early’ during spontaneous neutrophil apoptosis (See Table 3; UDP glycosyltransferase 8).

We also demonstrate the ‘early’ regulation during spontaneous neutrophil apoptosis, of an oxygen regulated protein (See Table 3); this has been shown to increased in astrocytes upon hypoxia and reoxygenation and proposed to be a molecular chaperpone to cope with environmental stress.

Lipoxygenase expression has been closely associated with cell growth inhibition and apoptosis. 15-lipoxygenase has recently, and since the initial submission of this patent, been suggested as a novel molecular target of nonsteroidal anti-inflammatory drugs (NSAIDS) for inducing apoptosis in colorectal carcinogenesis (Shureiqi I et al., 2000. J Natl Cancer Inst 92(14):1136-42). Also, recently, lipoxygenase ‘early’ activation has been identified as a key element in the execution of apoptosis induced by oxidative stress in plant cells (MacCarrone M et al., 2000. Eur J Biochem. 267(16):5078-84). We show here that both 12- and 15-lipoxygenase are regulated ‘early’ in neutrophil spontaneous apoptosis (See Table 3).

Gene Expression Changes Correlate Across Various Models That Share the Induction of Apoptosis by ROS.

The models of ROS-induced apoptosis are particularly effective as discovery tools to identify key regulators of the apoptosis process when ‘cluster analysis’ is combined across several complimentary experiments. For example BCL2/adenovirus E1B 19 kD-interacting protein 3-like mRNA (BNIP3L) is commonly associated with ‘early’ ROS-induced apoptosis in several of these models; including neutrophil spontaneous apoptosis, HeLa cell apoptosis induced by cisplatin and HeLa cell apoptosis induced by UV-irradiation (See Table 4). Similarly, retinoic acid receptor responder (tazarotene induced) 3 mRNA is commonly associated with ‘early’ ROS-induced apoptosis in several of these models; including neutrophil spontaneous apoptosis, HeLa cell apoptosis induced by cisplatin, HeLa cell apoptosis induced by UV-irradiation and HeLa cell apoptosis induced by treatment with TNFα and cycloheximide (See Table 4).

Genes Regulated ‘Early’ in Neutrophil Spontaneous Apoptosis are Identified by Suppression Subtractive Hybridisation.

This example describes the use of SSH, with modifications discussed, to identify and clone genes that are regulated ‘early’ in the process of spontaneous neutrophil apoptosis. Many of these DNA sequences are novel, i.e. they are not recognised by BLAST analysis to public gene sequence databases.

Following subtractions between the 0, 2 and 4 hour time points of neutrophil spontaneous apoptosis, a preliminary screening procedure is applied to arrays of clones generated from the subtracted libraries, (as described). Approximately 1000 clones are screened from each of the four subtracted libraries, yielding 700 differentially expressed clones. Of these clones, 423 are down-regulated as apoptosis proceeds, while 277 are up-regulated as apoptosis proceeds.

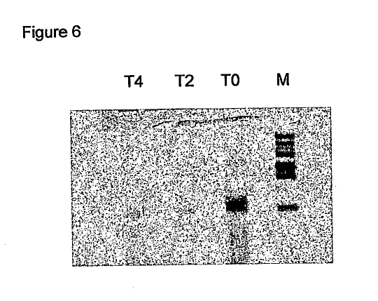

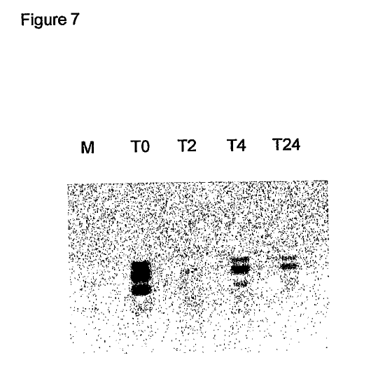

Of circa two hundred differentially expressed clones sequenced, approximately 30% of sequences share identity with well-characterised proteins in the GenBank database. A further 30% share identity with uncharacterised ESTs and an additional 30% are novel, having no identified human EST homologue (See FIGS. 6 and 7).

### EXAMPLE 2

Establishment and Characterisation of a ‘Model Cell-System/Discovery Assay’ to Study the Molecular Mechanisms of Cellular Response to ROS and Apoptosis, in Primary Human Neutrophils, By Inhibiting this Apoptosis By the Addition of the Growth/Survival Factor GM-CSF.

This example describes the establishment of a model cell system in primary human neutrophils. Neutrophil spontaneous apoptosis is delayed by the addition of the survival factor GM-CSF. This ‘Model Cell System’ forms the basis of a novel ‘Discovery Assay’ that is then used for the characterisation, and identification of nucleotide (genes, mRNAs) or polypeptide (proteins, peptides) sequences associated with and potentially responsible for the molecular mechanisms of cellular response to ROS and apoptosis, including; 1) the initiation of the apoptosis process, 2) the cellular signalling pathways, and 3) the cells attempt to combat ROS and apoptosis in the cell.

Dose Responsiveness of the Anti-Apoptotic Effect of GM-CSF.

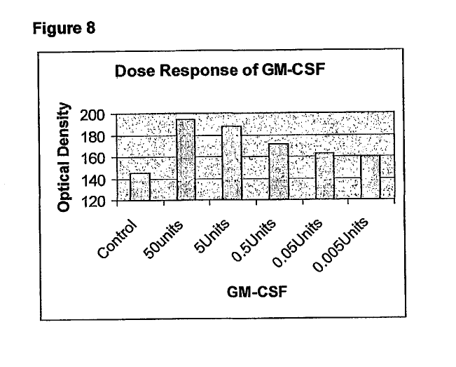

primary human neutrophils are isolated and purified from peripheral blood of normal healthy individuals. Neutrophils are resuspended in serum containing culture medium together with various amounts of GM-CSF at a density of 2×10⁶/ml, with 100 μl plated into a 96 well plate and cultured for 18 h at 37° C. After this time 10 μl of MTT (5 mg/ml) is added to the cultures and incubated for a further 4 h at 37° C. before solubilisation of the purple coloured formazan with acidic isopropanol. Optical densities are read at 570 nm using a plate reader. Our results demonstrate a direct correlation between survival and concentrations of GM-CSF added to the culture medium (FIG. 8).

Determination of the Temporal Relationship Between GM-CSF Addition and Survival

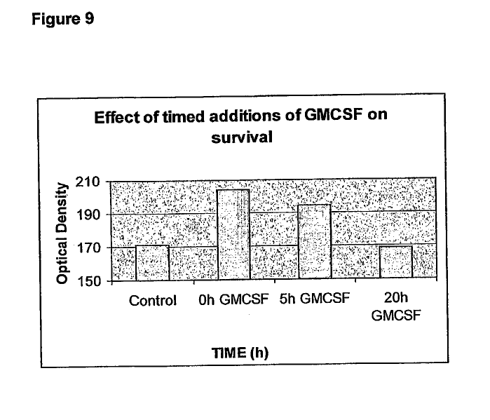

A time course of GM-CSF additions was carried out to neutrophils in order to determine up to what stage GM-CSF can induce survival in neutrophils. primary human neutrophils are isolated and purified from peripheral blood of normal healthy individuals. Neutrophils are resuspended in serum containing culture medium at a concentration of 2×10⁶/ml, with 100 μl/well plated into a 96 well plate and culture at 37° C. commenced. At the indicated time points GM-CSF (50U/ml) was added to the neutrophils and culture continued until 20 h post initiation of culture. After this time, 10 μl of MTT (5 mg/ml) was added to the cultures and incubated for a further 4 h at 37° C. before solubilisation of the purple coloured formazan with acidic isopropanol. Optical densities were read at 570 nm using a plate reader. Our results demonstrate that GM-CSF can be added to neutrophils as late as 5 hours post isolation and not get any discernible loss in the protective effect of this cytokine. However, if administration of the cytokine is delayed for 20 h post isolation, the protective effect is zero in 24 h cultures. This data indicates that the commitment to die for the vast majority of neutrophils is later than 5 hours post isolation (See FIG. 9). This result is in agreement with our other findings that morphological characteristics of apoptosis occurs between 6 and 8 hours.

Determination of GM-CSF “On Time” Sufficient for Apoptosis Delay

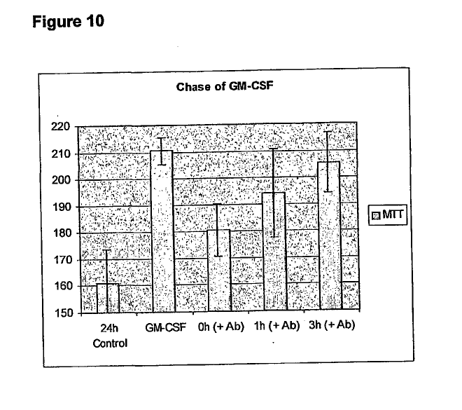

We performed timed additions of anti-GM-CSF antibody to neutrophils in order to determine how immediate the anti-apoptotic effects induced by GM-CSF occur. primary human neutrophils are isolated and purified from peripheral blood of normal healthy individuals. Neutrophils are resuspended in serum containing culture medium containing 5 U/ml of GM-CSF at a concentration of 2×10⁶/ml, with 100 wl/well plated into a 96 well plate and culture at 37° C. commenced. At the indicated time points additions 10 μg/ml anti-GM-CSF are made to the neutrophils and cultured for 18 h at 37° C. After this time, 10 μl of MTT (5 mg/ml) are added to the cultures and incubated for a further 4 h at 37° C. before solubilisation of the purple coloured formazan with acidic isopropanol. Optical densities are read at 570 nm using a plate reader. Comparable survival is obtained when addition of anti-GM-CSF is delayed for 3 hours following isolation as to when no neutralising antibody is added (See FIG. 10). In contrast, when neutralising antibody is added before 3 hours, survival is directly linked to the addition time. This indicates that the survival mediated by GM-CSF is not immediate and that cells must be exposed to the cytokine for a defined period of time (“on time”) before survival is conferred.

Demonstration that GM-CSF-Mediated Survival of the Neutrophil Does Not Significantly Operate Through the Glutathione System.

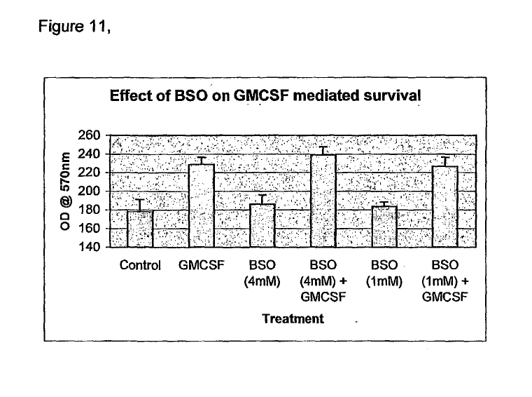

Spontaneous apoptosis is mediated through ROS. Intracellular concentrations of reduced glutathione maintains redox potential and thus protects the cell against oxidative damage, prolonging survival. We examined whether GM-CSF mediated survival is acting through increasing the glutathione concentration by examining the glutathione system. primary human neutrophils are isolated and purified from peripheral blood of normal healthy individuals. Neutrophils (2×10⁶) are resuspended in serum containing culture medium +/− GM-CSF (5 u/ml) and either with or without the gamma glutamylcysteine synthase inhibitor BSO (1 or 4 mM). Cells are plated 100 μl into a 96 well plate and cultured for 18 h at 37° C. After this time 10 μl of MTT (5 mg/ml) are added to the cultures and incubated for a further 4 h at 37° C. before solubilisation of the purple coloured formazan with acidic isopropanol. Our results demonstrate that inhibiting gamma glutamylcysteine synthase with BSO and consequently the ability of the cell to synthesise reduced glutathione, did not impact on GM-CSF to mediate survival, suggesting that GM-CSF is not acting through production of large amounts of reduced glutathione to maintain redox potential (See FIG. 11).

Determination of mRNA Levels of Key Enzymes in Glutathione Synthesis

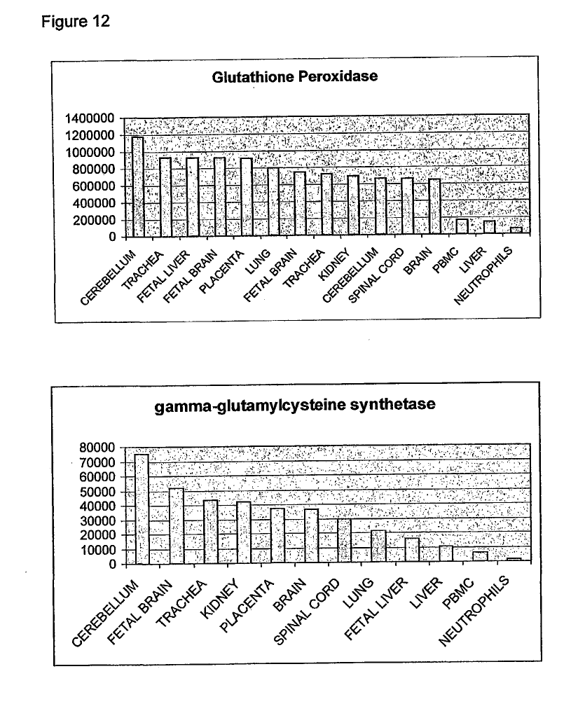

primary human neutrophils are isolated and purified from peripheral blood of normal healthy individuals. Simultaneously, peripheral blood mononuclear cells (PBMC) are purified from the same blood. Total cellular RNA is extracted from both neutrophil and PBMC and this is compared to commercial RNA (Clontech) isolated from a range of human tissues for levels of expression of RNA for key enzymes in the glutathione system; using the Incyte “LifeGrid”. Levels of expression of Glutathione Peroxidase and γ-glutamylcysteine synthase are shown in FIG. 12. There is a dramatic decrease in the level of transcription for both of these enzymes in liver, PBMC and neutrophils relative to other tissue As can be seen, transcription rates are lowest in neutrophil RNA. This is in agreement with other workers ( Rollet-Labelle E, Grange M J, Elbim C, Marquetty C, Gougerot-Pocidalo M A, Pasquier C 1998 Hydroxyl radical as a potential intracellular mediator of polymorphonuclear neutrophil apoptosis. Free Radic Biol Med 24(4): pp563-72) who demonstrated that neutrophils had approximately three fold lower expression of glutathione than PBMC. This further supports our contention that the glutathione system is not very active in the neutrophil.

GM-CSF-Mediated Neutrophil Protection is Associated With Significant Changes in Gene Expression.

Primary human neutrophils are isolated and purified from peripheral blood of normal healthy individuals. Neutrophils are resuspended in serum containing culture medium together with GM-CSF (50U) at a concentration of 2×10⁶/ml, and cultured for 0 h (control), 2 h, 4 h and 6 h at 37° C. After these times, total cellular RNA is extracted and this used as the starting material for analysis of gene expression changes.

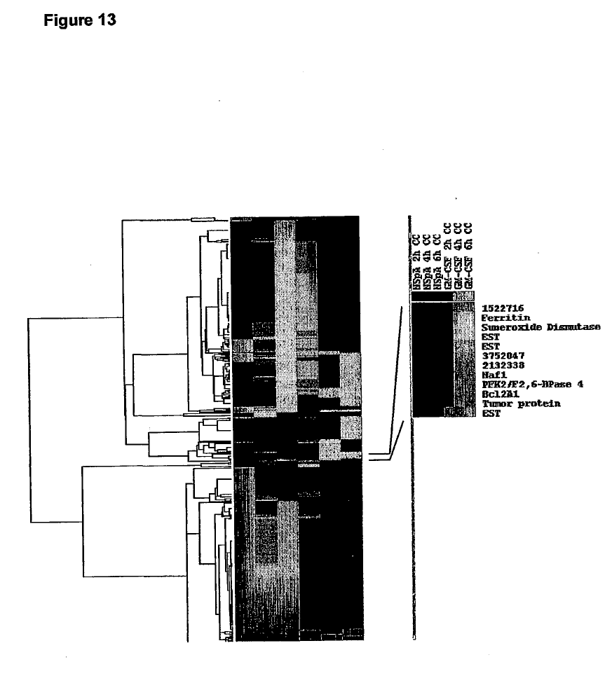

Significant changes in gene expression are measured following GM-CSF treatment, and associated with inhibition of neutrophil spontaneous apoptosis. Of ˜8000 human genes represented on commercial Incyte ‘LifeGrid’ microarray filters, greater than 360 genes change expression >2-fold with respect to the control 0 h isolated neutrophils. FIG. 13 shows these changes after cluster analysis. A significant number of genes are regulated as early as 2 h post-isolation of neutrophils; with ˜230 genes rapidly down-regulated and 16 genes up-regulated. Up-regulated genes increase in number progressively with time; 65 genes are up-regulated 4 h post-isolation and 155 genes are up-regulated 6 h post-isolation.

Cluster-Analysis: GM-CSF-Mediated Neutrophil Protection is Associated With Increase in Gene Expression of Enzymes That Degrade O₂⁻

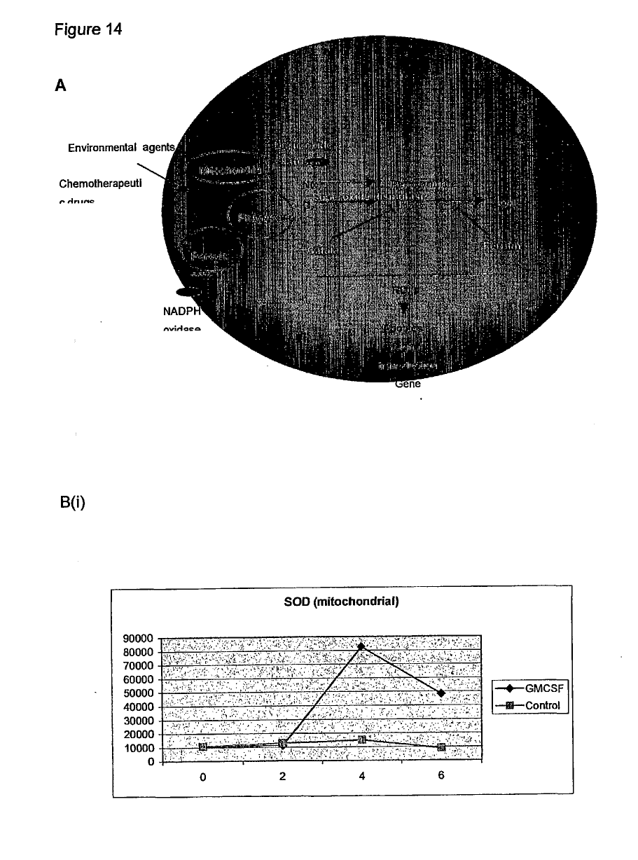

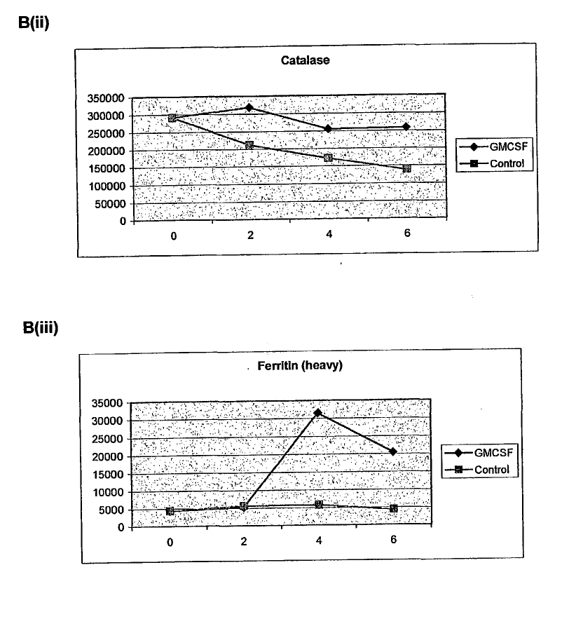

Primary human neutrophils are isolated and purified from peripheral blood of normal healthy individuals. Neutrophils are resuspended in serum containing culture medium together with GM-CSF (50U) at a concentration of 2×10⁶/ml, and cultured for 0 h (control), 2 h, 4 h and 6 h at 37° C. After these times, total cellular RNA is extracted and this used as the starting material for analysis of gene expression changes using the Incyte “LifeGrid” microarray. Following cluster analysis, a cluster of genes are identified that contained several genes involved in neutrophil survival and whose transcription is up—regulated upon GM-CSF treatment. This cluster, which included BcI2-A1 (a ‘known’ antiapoptotic neutrophil BCL2 family member) is termed the ‘survival’ cluster (See FIG. 14). Among these genes, one group that could be clearly identified are those genes whose protein products are involved in protecting the cell against an increased oxidative environment (FIG. 14). Thus GM-CSF increased expression of mitochondrial superoxide dismutase (dismutases superoxide to hydrogen peroxide), catalase (converts H₂O₂ to H₂O and O₂) and ferritin (which sequesters iron thus limiting the Fenton reaction and preventing production of hyper-reactive hydroxyl radical) above control levels and this coincided with the ability of GM-CSF to prolong survival in neutrophils. Since all these gene products are attenuators of the oxidative cellular response it is indicative that GM-CSF protects the cell by reducing the harmful effects of reactive oxygen species. The corollary therefore is that spontaneous apoptosis must be due to an uncontrolled oxidative environment within the cell. Furthermore, this reaffirms that the neutrophils response to reactive oxidative species is independent of the glutathione system.

GM-CSF Mediated Neutrophil Protection is Associated With Increase in Gene Expression Associated With the Pentose Phosphate Pathway

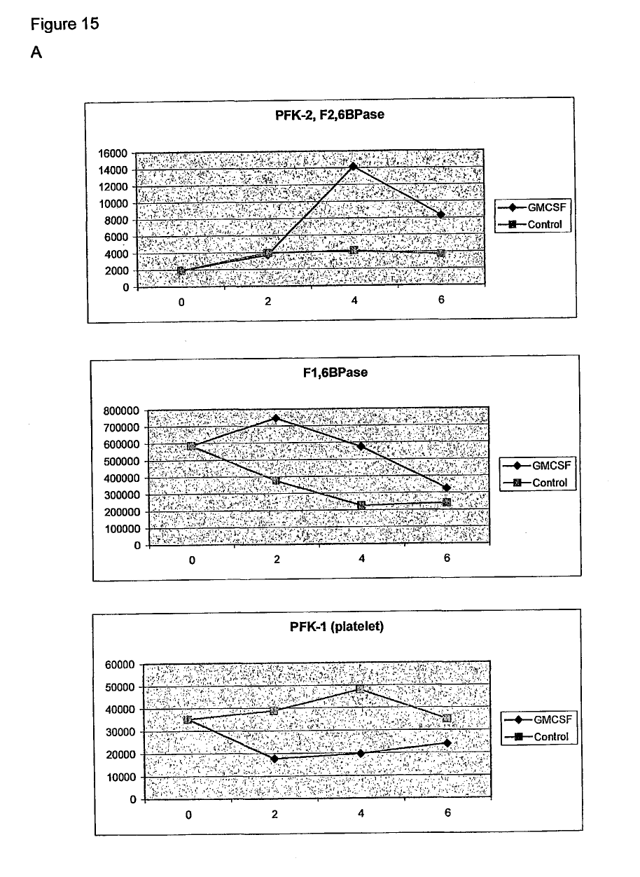

On further analysis of this survival cluster, we identified increased transcription of the bifunctional enzyme phosphofructokinase-2 (PFK-2)/fructose 2,6-bisphosphatase (FBPase-2) which regulates the cellular concentration of fructose 2,6-bisphosphate and which in turn can regulate the function of phosphofructokinase-1 (See FIG. 15). The ability of a cell to be able to reduce its oxidative environment is contingent on the cells ability to produce NADPH. This is particularly important in tissues that carry out the biosynthesis of fatty acids such as the liver and mammary tissue and other cells that are subjected to oxidative stress (Voehringer D. W., Hirschberg D. L., Xiao J., Lu Q., Roederer M., Lock, C. B. Herzenberg L. A., Steinman L., Herzenberg, L. A. (2000) PNAS, 97(6) pp2680-2685). Consequently, genes, which are represented on the array and whose products are known to be important in the pentose phosphate pathway are examined.

GM-CSF upregulates fructose 1,6 bisphosphatase (which functions to convert fructose 1,6 bisphosphate to fructose 6 phosphate which can then reversibly convert to Glucose-6-phosphate, the primary substrate for the pentose phosphate pathway. Simultaneously with up regulation of fructose 1,6 bisphosphatase, GM-CSF down regulated the transcription of phosphofructokinase 1 (whose role is to convert fructose 6 phosphate to fructose 1,6 bisphosphate), again ensuring that the pentose phosphate pathway is favoured above glycolysis. Therefore it is concluded that GM-CSF increases the formation of glucose 6 phosphate which can then be utilised in generation of reducing NADPH via the pentose phosphate pathway.

### EXAMPLE 2

Establishment and Characterisation of a ‘Model Cell-System/Discovery Assay’ to Study the Molecular Mechanisms of Cellular Response to Endogenous Induction of ROS and Apoptosis, in HeLa Cells, by Exogenous Addition of Cisplatin (‘Induced Apoptosis’).

This example describes the establishment of a model cell system in HeLa cells. The culture of HeLa cells and treatment with cisplatin is described. This ‘Model Cell System’ forms the basis of a novel ‘Discovery Assay’ that is then used for the characterisation, identification and functional validation of nucleotide (genes, mRNAs) or polypeptide (proteins, peptides) sequences associated with and potentially responsible for the molecular mechanisms of cellular response to ROS and apoptosis, including; 1) the initiation of the apoptosis process, 2) the cellular signalling pathways, and 3) the cells attempt to combat ROS and apoptosis in the cell.

The platinum compound cisplatin (cis-diamminedichloroplatinum (II), CDDP) is among the most widely used cytotoxic anticancer drug. Intemucleosomal DNA cleavage and ultrastructural changes characteristic of apoptosis have been observed following cisplatin exposure (Barry M A, Behnke C A, Eastman, A. (1990) Activation of programmed cell death (apoptosis) by cisplatin, other anticancer drugs, toxins, and hyperthermia. Biochem Pharmacol 40, pp2353-62). The specific mechanism of tumour death following cisplatin exposure has been reported to be due to the drug inducing endogenous ROS-induced stress; (Miyajima A, Nakashima J, Yoshioka K, Tachibana M, Tazaki H, Murai M (1997) Role of reactive oxygen species in cis-dichlorodiammineplatinum-induced cytotoxicity on bladder cancer cells. Br J Cancer 76 (2) pp206-10

We demonstrate the utility of this model system to ‘discover’ early regulated genes associated with ROS-induced apoptosis, by the discovery of genes that have ‘known’ involvement in cell apoptosis, carcinogenesis, signal transduction and the cellular response to ROS, oxidative and environmental stress. In many cases these examples include genes, identified by cluster analysis within and between different ROS-induced apoptosis models, that share coordinate regulation and which belong to the same signal transduction pathways or cellular processes. Similarly we demonstrate the identification of genes regulated ‘early’ in ROS-induced apoptosis that have no known biological function i.e. ESTs from both commercial filters. By association with known genes, as described, these novel genes themselves are identified as candidate apoptosis genes that may similarly function in 1) the initiation of the apoptosis process, 2) the cellular signalling pathways, and 3) the cells attempt to combat ROS and apoptosis in the cell.

Determination of Viability Using a MTT Assay

Culture viability is determined using an MTT (3-[4, 5-Dimethylthiazol-2-yl]-2, 5-diphenyltetrazolium bromide) assay. The assay is based on the principle that MTT is a water soluble tetrazolium salt. Dissolved MTT is converted to an insoluble purple formazan by cleavage of the tetrazolium ring by active mitochondria of living cells but not of dead cells.

In our system, cells are cultured in 96 well plates in 100 μl of culture medium and are treated in ways known to induce apoptosis. Following an indicated period of time, 10 μl MTT stock solution (5 mg/mi) is added to each culture being assayed and incubated at 37° C. for 4 h. At the end of the incubation period the converted dye is solubilised with acidic-isopropanol (0.04N HCL. Absorbance of converted dye is measured at a wavelength of 570 nm.

Determination of Viability Using a Crystal Violet Assay

The following example describes a method to analyse viability of adherent cells, such as HeLa, using chemical stain Crystal Violet. Adherent cells are cultured in the presence of known agents to induce/ modulate the apoptotic process. After a suitable period of time, supernatants are removed by inverting and flicking the plate. Wells are washed twice with PBS and remaining adherent cells are fixed with 100% EtOH and air-dried. To each well is added 100 μl of 1% aqueous Crystal Violet Solution (Sigma). Plates are then incubated at room temperature (RT) for 5 min before washing in tap water. Wells are solubilised in 33% acetic acid and optical density is measured at 570 nm using a plate reader.

Cisplatin Cytotoxicity is Dose Responsive

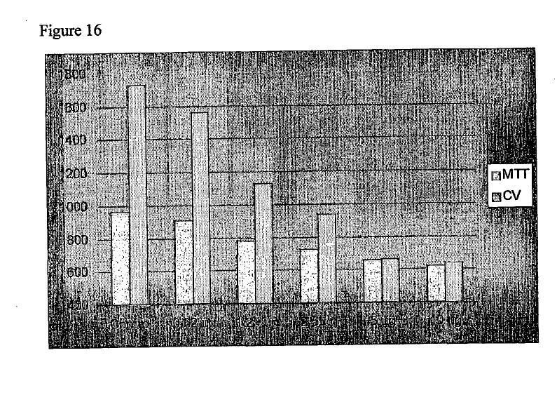

HeLa cells are plated in full culture medium at a concentration of 2×10⁴ cells/well of 96 well plate. Cells are allowed to adhere for 4 hours before replacing the medium with fresh medium contain cisplatin at the indicated dose range. Cells are cultured overnight at 37° C. before analysing for apoptosis using either MTT or crystal violet. As can be seen in FIG. 16, HeLa cell death is dose dependent on the level of cisplatin present in the medium

Cisplatin Treatment Induces Apoptosis as Measured by DNA Fragmentation in HeLa

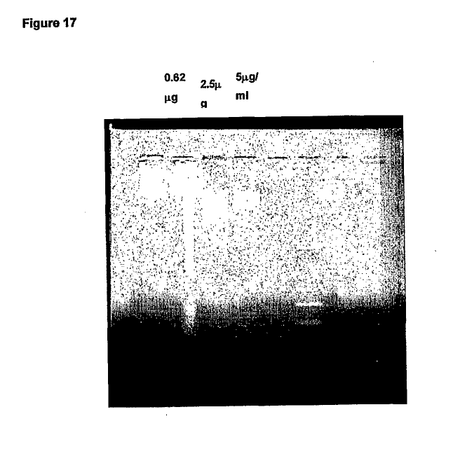

HeLa cells are plated in full culture medium at a concentration of 5×10⁵ cells/well of 24 well plate. Cells are allowed to adhere for 4 hours before replacing the medium with fresh medium contain cisplatin at the indicated dose range. Cells are cultured overnight at 37° C. before being lysed (5×10⁶/treatment) in lysis buffer (1M Tris, 10 mM EDTA , 0.8% sarcosine L sulphate) and incubated on ice for 15 min before centrifugation at 12000g. Supernatant is removed and phenol/chloroform extracted. Following precipitation of DNA, pellets are resuspended in Tris/EDTA buffer (TE) containing 100 μg/ml RNAase A and incubated for 2 h @37° C. Proteinase K (100 μg/ml) is then added and sample incubated for a further 18 h before 1 g of DNA is separated on a 2% agarose gel. FIG. 17 represents a DNA fragmentation gel following treatment of HeLa cells with differing concentrations of cisplatin. As can be seen, samples that received cisplatin display a laddering effect typical of apoptosis and in contrast to control cells where a singular heavy band is seen.

Modulation of Cisplatin Induced Apoptosis by Agents that Impact the Oxidative Environment; Demo that Cisplatin Acting Through ROS (BSO/NAC)

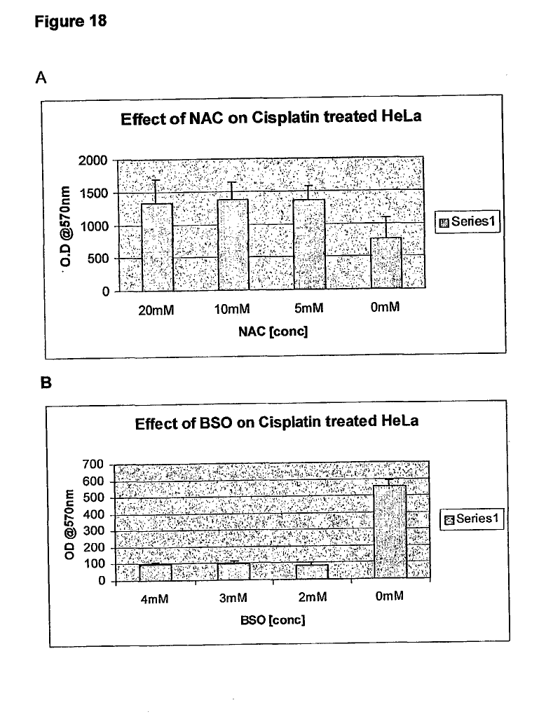

HeLa cells are plated in full culture medium at a concentration of 2×10⁴ cells/well of 96 well plate. Cells are allowed to adhere for 4 hours before replacing the medium with fresh medium containing cisplatin (10 μg/ml) and either NAC (which increases levels of glutathione) or BSO (which inhibits γ-glutamylcysteine synthase- thus preventing synthesis of glutathione) at the indicated dose range. Cells are cultured overnight at 37° C. before analysing for apoptosis using crystal violet. As can be seen in FIG. 18, HeLa cell sensitivity to cisplatin is decreased when NAC is present in the medium. In contrast, the presence of BSO seems to increase the sensitivity of HeLa cells towards cisplatin. The ability to modulate the sensitivity of HeLa cells towards cisplatin with reagents which alter intracellular glutathione levels and as such modify the oxidative environment confirms a role for reactive oxygen species

Cisplatin-Induced HeLa Cell Apoptosis is Associated With Significant Changes in Gene Expression.

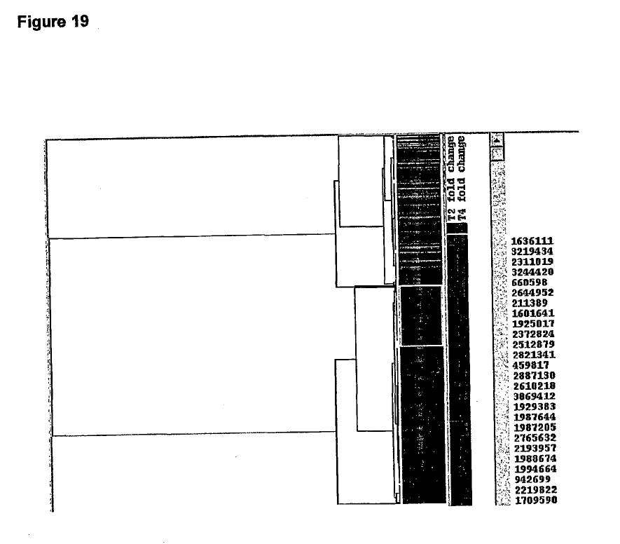

HeLa cells are maintained in culture, and treated with cisplatin (10 μg/ml). At various time points, total cellular RNA is isolated and examined for gene expression changes using microarray. Significant transcription of mRNA is observed. In one example experiment, as many as 2400 genes are detected using Incyte human ‘LifeGrid’ filters; these genes are increased or decreased by greater than 2-fold over a 4-hour time course as compared with the time zero control HeLa cells. As many as 2000 genes are increased or decreased as early as 2 hours post-isolation. FIG. 19 shows cluster analysis of genes regulated during cisplatin-induced HeLa apoptosis time course. Genes fall into primary clusters of up (red) and down-regulated (green) genes.

Many of these regulated genes are ESTs (˜350) with no known biological functional annotation. Many more (data not shown) have only very limited biological characterisation.

Changes in ‘Early’ Gene Expression Include Classes of Genes With ‘Known’ Roles in Apoptosis.

Of those genes regulated as early as 2 hours post-isolation, classical apoptosis-related genes including both caspase and BcI-2 related genes are detected (See Table 5). Caspase 3, 6, 8 and 9 are regulated. BCL2/adenovirus E1B 19 kD-interacting protein 3-like is regulated, and this is also described in the example for neutrophil spontaneous apoptosis. In addition, BCL2, BCL2-associated athanogene, apoptosis inhibitor 1 and apoptosis inhibitor 4 (survivin) are also regulated.

In addition, we show that CASP2 and RIPK1 domain-containing adaptor with death domain (CRADD) is regulated ‘early’ in cisplatin-induced HeLa apoptosis (See Table 5). CRADD (otherwise known as RAIDD) has been shown to act as an adaptor molecule in recruiting the death protease caspase 2 to the TNF receptor 1 signalling complex. Its overexpression in mammalian cells induced apoptosis (Duan, H.; Dixit, V. M.: RAIDD is a new ‘death’ adaptor molecule. Nature 385: 86-89, 1997).

Changes in ‘Early’ Gene Expression Include Genes Associated Cellular Response to ROS and Oxidative/Environmental Stress.

The following observations support and confirm a key ‘early’ regulatory role for ROS and oxidative stress in neutrophil spontaneous apoptosis, and in the regulation of ROS-related gene expression.

Lipoxygenase expression has been closely associated with cell growth inhibition and apoptosis, as described for neutrophil spontaneous apoptosis, and has been identified as a key element in the execution of apoptosis induced by oxidative stress in plant cells (MacCarrone M et al., 2000. Eur J Biochem. 267(16):5078-84). We show here that both 5- and 12-lipoxygenase are regulated ‘early’ in cisplatin-induced HeLa apoptosis.

Cigarette smoke, containing reactive oxygen species, is the most important risk factor for chronic pulmonary emphysema (CPE). Heme-oxygenase plays a protective role as an antioxidant in the lung. Yamada et al analysed the promoter activities of the heme oxygenase gene carrying different (GT)n repeats by transient-transfection assays in cultured cell lines. The smaller class alleles showed greater upregulation of transcriptional activity on H₂O₂ exposure. These findings suggested that the large size of a (GT)n repeat in the heme oxygenase gene promoter may reduce heme oxygenase inducibility by reactive oxygen species in cigarette smoke, thereby resulting in the development of CPE Yamada, N.; Yamaya, M.; Okinaga, S.; Nakayama, K.; Sekizawa, K.; Shibahara, S.; Sasaki, H. : Microsatellite polymorphism in the heme oxygenase-1 gene promoter is associated with susceptibility to emphysema. Am. J. Hum. Genet 66: 187-195, 2000). We show here that heme oxygenase is regulated ‘early’ in cisplatin-induced HeLa apoptosis (See Table 5).

As described for neutrophil spontaneous apoptosis, two key ‘known’ transcription regulators of the cellular response to oxidative stress are hypoxia-inducible factor 1 (HIF-1) and arylhydrocarbon receptor (AHR) (Morel, Y and Barouki, R. 1999. Repression of gene expression by oxidative stress. Biochem J. 342:481-496). We show here that the aryl hydrocarbon receptor nuclear translocator and a homologue aryl hydrocarbon receptor nuclear translocator-like mRNAs are both regulated ‘early’ in cisplatin-induced HeLa apoptosis (See Table 5). Similarly, hypoxia-inducible factor 1-alpha is also regulated ‘early’ in cisplatin-induced HeLa apoptosis.

Iron-responsive element (IRE) binding protein (IRP1) plays an important role in iron metabolism. IRP1 appears to be a direct target for ROS (Morel, Y and Barouki, R. 1999. Repression of gene expression by oxidative stress. Biochem J. 342:481-496). Several studies have reported that the oxidation of IRP1 by ROS, including exogenous addition of H₂O₂, regulate its activity. When IRP1 binds Ferritin mRNA its translation is repressed. The regulation and importance of ferritin for the detoxification of H₂O₂ in cells is discussed here in the example for inhibition of spontaneous neutrophil apoptosis by GM-CSF. We show here that IRP1 mRNA is also regulated ‘early’ in cisplatin-induced HeLa apoptosis (See Table 5).

One of the most well characterised transcription factors that is regulated by ROS is NFKB. Not only is NFicB well established in its known regulation by ROS e.g. H₂O₂, but it also has a key role in both apoptosis and cell survival (Morel, Y and Barouki, R. 1999. Repression of gene expression by oxidative stress. Biochem J. 342:481-496). We show here that NFκB, subunit 2, is regulated ‘early’ in cisplatin-induced HeLa apoptosis (See Table 5).

Gene Expression Changes Correlate Across Various Models That Share the Induction of Apoptosis by ROS.

The models of ROS-induced apoptosis are particularly effective as discovery tools to identify key regulators of the apoptosis process when ‘cluster analysis’ is combined across several complimentary experiments. For example, greater than 1000 of the same genes are regulated (increased or decreased 2-fold or more relative to control untreated cells) when comparing HeLa cells treated with cisplatin to HeLa cells treated with UV-irradiation; both go through an ROS-mechanism (See also Example 4).

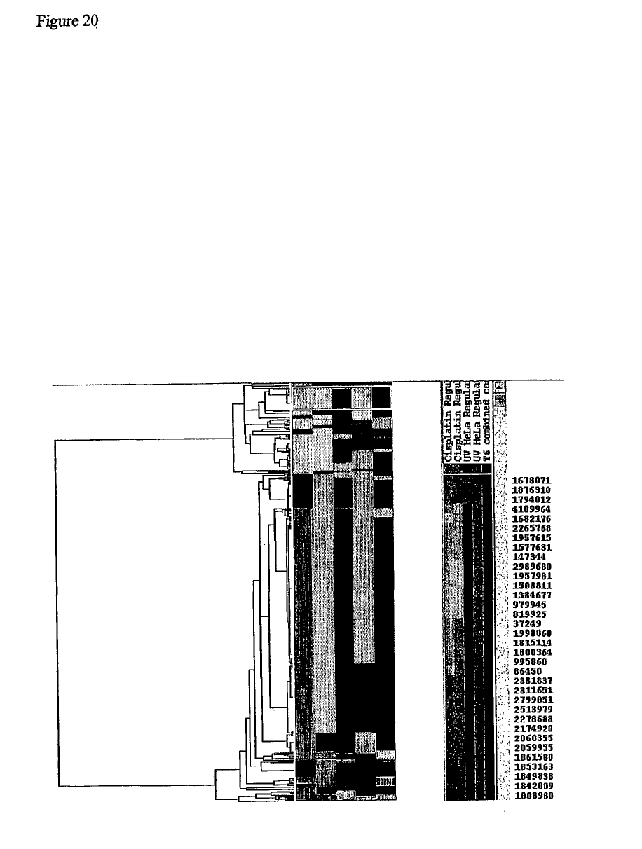

FIG. 20 shows gene expression changes in HeLa cells treated with cisplatin to HeLa cells treated with UV-irradiation, when analysed by ‘cluster analysis’. A cluster is illustrated, of gene who's expression of mRNA is increased ‘early’, from 2 hours, both with UV-irradiation and also with cisplatin exposure. Many of these genes are ESTs with little of no known function, but a significant portion of them are ‘known’ genes that are directly or indirectly involved in apoptosis or the cell defence mechanisms to ROS and drug resistance (See FIG. 20). Of particular note is a gene previously described as ‘cisplatin resistance associated’. Caspase 3, myc and RAS p21 protein activator are well ‘known’ apoptosis mediators. The following 8 genes have been described in the context of cellular ROS and drug resistance: 1) tyrosine 3/tryptophan 5-monooxygenase activation protein is a 143-3 protein; the 14-3-3 proteins promote survival by disrupting binding of BAD to prosurvival BCL2 proteins (Mol Cell 2000 6(1):41-51, 2) rTS beta protein is a component of tymidylate synthase, associated with 5FU and Tomudex drug resistance (Br J Cancer 1999 81(2):252-60, 3) UDP glycosyltransferase 2; glycosyltransferases are associated with protection from quinone induced apoptosis (Toxicol AppI Pharmacol 2000 162(1) 34, 4) aminopeptidase puromycin sensitive; aminopeptidaseis associated with resistance to UV-irradiation (Mutat Res 1998 422(1):55-67, 5) proteasome 26S and 6) 26S proteasome-associated pad1 homolog; overexpression of 26 proteasome subunit associated with resistance to diverse drugs and UV-irradiation (J Biol Chem 1997 272(48):30470-5, 7) sulfotransferase family 2A, dehydroepiandrosterone (DHEA)—preferring, member 1; one of the major roles of sulfotransferases is in the metabolism of drugs, and 8) tetraspan 1; recently, and since submission of this patent, Voehringer et al reported that mouse B cell lymphoma cells express 20 to 50 fold increased mRNA for tetraspanin associated with UV-irradiation (PNAS 2000 97(6) 2680-2685). These results strongly demonstrate the utility of combining these models with cluster analysis of global gene expression patterns to identify genes associated with ROS-induced apoptosis and cell defence.

### EXAMPLE 4

Establishment and Characterisation of a ‘Model Cell-System/Discovery Assay’ to Study the Molecular Mechanisms of Cellular Response to ROS and Apoptosis, in HeLa Cells, by Exogenous Treatment With UV-Irradiation (‘Induced Apoptosis’).

This example describes the establishment of a model cell system in HeLa cells. The culture of HeLa cells and treatment with UV-irradiation is described. This ‘Model Cell System’ forms the basis of a novel ‘Discovery Assay’ that is then used for the characterisation, identification and functional validation of nucleotide (genes, mRNAs) or polypeptide (proteins, peptides) sequences associated with and potentially responsible for the molecular mechanisms of cellular response to ROS and apoptosis, including; 1) the initiation of the apoptosis process, 2) the cellular signalling pathways, and 3) the cells attempt to combat ROS and apoptosis in the cell.

Exposure of mammalian cells to ultraviolet (UV) light provokes oxidative stress by generating intracellular reactive oxygen species which in turn results in apoptosis (Chan, W-H, Yu, J-S (2000) Inhibition of UV Irradiation-induced oxidative stress and apoptotic biochemical changes in human epidermal carcinoma A431 cells by genestein. J. Cell Biochem. 78 pp73-84).

We demonstrate the utility of this model system to ‘discover’ early regulated genes associated with ROS-induced apoptosis, by the discovery of genes that have ‘known’ involvement in cell apoptosis, carcinogenesis, signal transduction and the cellular response to ROS, oxidative and environmental stress. In many cases these examples include genes, identified by cluster analysis within and between different ROS-induced apoptosis models, that share coordinate regulation and which belong to the same signal transduction pathways or cellular processes. Similarly we demonstrate the identification of genes regulated ‘early’ in ROS-induced apoptosis that have no known biological function i.e. ESTs from both commercial filters. By association with known genes, as described, these novel genes themselves are identified as candidate apoptosis genes that may similarly function in 1) the initiation of the apoptosis process, 2) the cellular signalling pathways, and 3) the cells attempt to combat ROS and apoptosis in the cell.

UV-Irradiation-Induced HeLa Cell Apoptosis

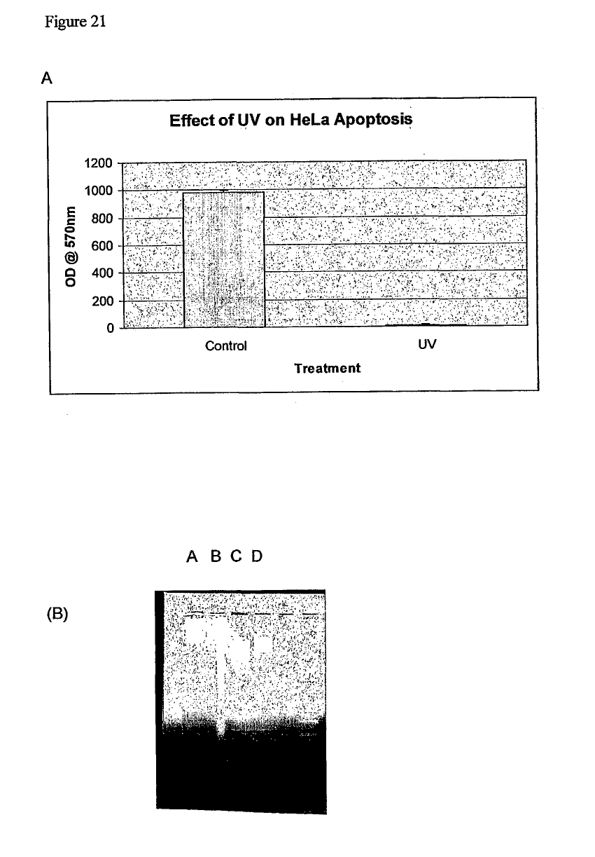

HeLa cells are plated into 96 well plate at a concentration of 2×104/well. After cells are allowed to adhere for 4 hours, a UV-irradiation insult is delivered by placing plates into a Hoefer UV crosslinker and exposing the cells to 1.2 joules/cm2 for 3 mins. Control plates are plated simultaneous with the UV insulted but are not exposed. Cells are returned to culture @37° C. overnight. Cell viability is determined by crystal violet staining and reading optical density @570 nm. As represented in FIG. 21, UV irradiation induced close to 100% apoptosis.

UV-Irradiation-Induced HeLa Cell Apoptosis is Associated With Significant Changes in Gene Expression.

HeLa cells are plated at a concentration of 5×10⁵ cells/ml of 24 well plate and allowed to adhere for 4 hours before being exposed to 1.2 j/cm² for 3 mins using a Hoefer UV crosslinker. Following insult, cells are incubated at 37° C. At various time points, total cellular RNA from 5×10⁶ HeLa cells is isolated and examined for gene expression changes using microarray. Significant transcription of mRNA is observed. In one example experiment, as many as 1700 genes are detected using Incyte human ‘LifeGrid’ filters; these genes are increased or decreased by greater than 2-fold over a 24-hour time course as compared with the time zero control HeLa cells. As many as 160 genes are increased or decreased as early as 2 hours post-isolation.

Many of these regulated genes are ESTs (˜280) with no known biological functional annotation. Many more (data not shown) have only very little biological characterisation.

Changes in ‘early’ Gene Expression Include Genes Associated Cellular Response to ROS and Oxidative/Environmental Stress.

The following observations support and confirm a key ‘early’ regulatory role for ROS and oxidative stress in neutrophil spontaneous apoptosis, and in the regulation of ROS-related gene expression (See Table 7).

### EXAMPLE 5

Establishment and Characterisation of a ‘Model Cell-System/Discovery Assay’ to Study the Molecular Mechanisms of Cellular Response to ROS and Apoptosis, in HeLa Cells, by Exogenous Treatment With TNFα and Cycloheximide (‘Induced Apoptosis’).

This example describes the establishment of a model cell system in HeLa cells. The culture of HeLa cells and treatment with TNFα and cycloheximide is described. This ‘Model Cell System’ forms the basis of a novel ‘Discovery Assay’ that is then used for the characterisation, identification and functional validation of nucleotide (genes, mRNAs) or polypeptide (proteins, peptides) sequences associated with and potentially responsible for the molecular mechanisms of cellular response to ROS and apoptosis, including; 1) the initiation of the apoptosis process, 2) the cellular signalling pathways, and 3) the cells attempt to combat ROS and apoptosis in the cell.

A combination of TNFα and cycloheximide is known to induce apoptosis whereas either reagent alone will not. Moreover, the ability of TNFα to induce apoptosis is mediated via ROS (Goossens V, De Vos K, Vercammen D, Steemans M, Vancompemolle K, Fiers W, Vandenabeele P, Grooten J Redox regulation of TNF signalling. Biofactors 1999; 10(2-3):145-56).

We demonstrate the utility of this model system to ‘discover’ early regulated genes associated with ROS-induced apoptosis, by the discovery of genes that have ‘known’ involvement in cell apoptosis, carcinogenesis, signal transduction and the cellular response to ROS, oxidative and environmental stress. In many cases these examples include genes, identified by cluster analysis within and between different ROS-induced apoptosis models, that share coordinate regulation and which belong to the same signal transduction pathways or cellular processes. Similarly we demonstrate the identification of genes regulated ‘early’ in ROS-induced apoptosis that have no known biological function i.e. ESTs from both commercial filters. By association with known genes, as described, these novel genes themselves are identified as candidate apoptosis genes that may similarly function in 1) the initiation of the apoptosis process, 2) the cellular signalling pathways, and 3) the cells attempt to combat ROS and apoptosis in the cell.

Treatment of HeLa Cells With TNFα/Cycloheximide Induces Apoptosis as Measured by Cell Death

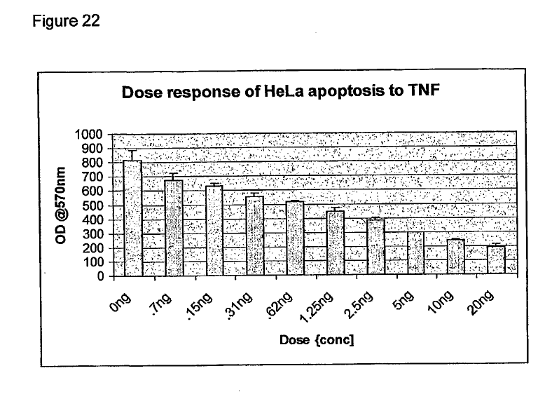

HeLa cells are plated into 96 well plate at a concentration of 2×10⁴/well and allowed to adhere for 4 hours Culture medium is then replaced with fresh medium containing the indicated concentrations of TNFα together with 5 μg/ml cycloheximide. Cultures are maintained for a further 18 hours, before measuring apoptosis with crystal violet and reading @570 nm in a plate reader. FIG. 22 shows that there is a dose. response between the concentration of TNFA used and the amount of apoptosis recorded for the HeLa cells.

Treatment of HeLa Cells With TNFα/Cycloheximide Induces Apoptosis as Measured by DNA Fragmentation

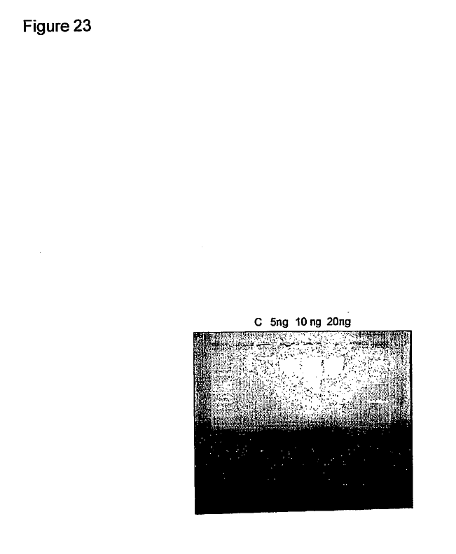

HeLa cells are plated into 24 well plate at a concentration of 5×10⁵/well and allowed to adhere for 4 hours Culture medium is then replaced with fresh medium containing the indicated concentrations of TNFα together with 5 μg/ml cycloheximide. Cultures are maintained for a further 18 h, before isolation of DNA from 5×10⁶ cells/treatment (as previously described) and running on a 2% gel. FIG. 23 shows resulting gel after separation. From the figure it is clear that TNFα/Chx treatment results in DNA laddering, which is characteristic of cells undergoing apoptosis.

TNFα/CHX-Induced HeLa Cell Apoptosis is Associated With Significant Changes in Gene Expression.

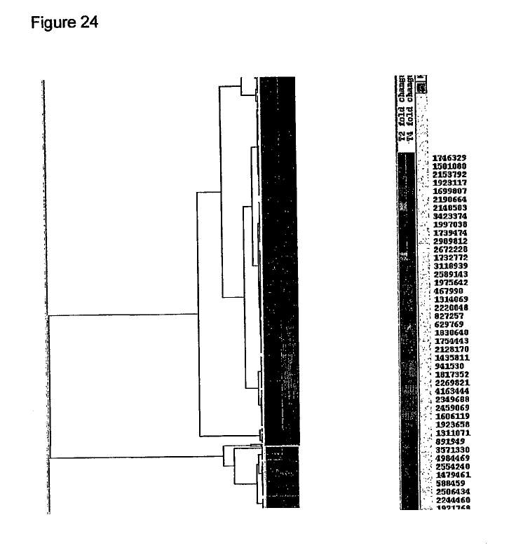

HeLa cells are maintained in culture, and treated with TNFα and cycloheximide. At various time points, total cellular RNA is isolated and examined for gene expression changes using microarray. Significant transcription of mRNA is observed. In one example experiment, as many as 420 genes are detected using Incyte human ‘LifeGrid’ filters; these genes are increased or decreased by greater than 2-fold over a 4-hour time course as compared with the time zero control HeLa cells. As many as 50 genes are increased or decreased as early as 2 hours post-isolation. FIG. 24 shows cluster analysis of genes regulated during UV-irradiation-induced HeLa apoptosis time course. Genes fall into primary clusters of up (red) and down- regulated (green) genes.

Many of these regulated genes are ESTs (˜60) with no known biological functional annotation. Many more (data not shown) have only very little biological characterisation.

### EXAMPLE 6

Establishment and Characterisation of a ‘Model Cell-System/Discovery Assay’ to Study the Molecular Mechanisms of Cellular Response to ROS and Apoptosis, in HL60 Cells, by Exogenous Addition of H₂O₂ (‘Induced Apoptosis’).

This example describes the establishment of a model cell system in HL60 cells. The culture of HL60 cells and treatment with exogenous ROS, in the form of H₂O₂ is described. This ‘Model Cell System’ forms the basis of a novel ‘Discovery Assay’ that is then used for the characterisation, identification and functional validation of nucleotide (genes, mRNAs) or polypeptide (proteins, peptides) sequences associated with and potentially responsible for the molecular mechanisms of cellular response to ROS and apoptosis, including; 1) the initiation of the apoptosis process, 2) the cellular signalling pathways, and 3) the cells attempt to combat ROS and apoptosis in the cell.

We demonstrate the utility of this model system to ‘discover’ early regulated genes associated with ROS-induced apoptosis, by the discovery of genes that have ‘known’ involvement in cell apoptosis, carcinogenesis, signal transduction and the cellular response to ROS, oxidative and environmental stress. In many cases these examples include genes, identified by cluster analysis within and between different ROS-induced apoptosis models, that share coordinate regulation and which belong to the same signal transduction pathways or cellular processes. Similarly we demonstrate the identification of genes regulated ‘early’ in ROS-induced apoptosis that have no known biological function i.e. ESTs from both commercial filters and from our own libraries of differentially expressed genes isolated by Suppression Subtractive

Hybridisation (SSH). By association with known genes, as described, these novel genes themselves are identified as candidate apoptosis genes that may similarly function in 1) the initiation of the apoptosis process, 2) the cellular signalling pathways, and 3) the cells attempt to combat ROS and apoptosis in the cell.

H₂O₂ Treatment Induces Apoptosis in HL-60 Cells, as Assessed by DNA Fragmentation ELISA

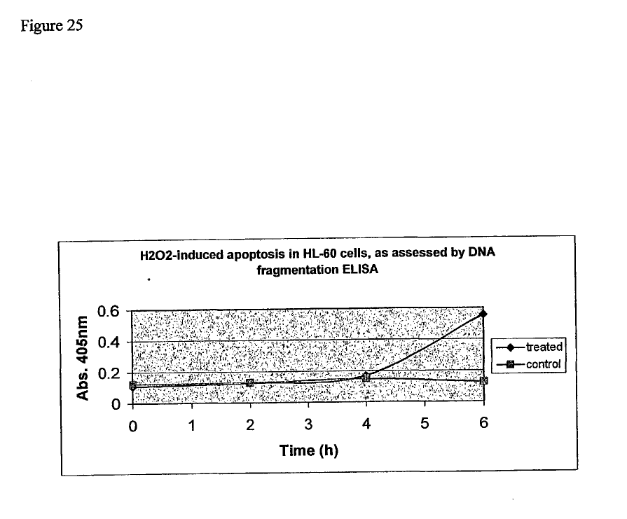

HL60 cell apoptosis is measured using a Cellular DNA fragmentation ELISA (Bohringer). HL-60 cells (5×10⁶/ml) are labelled for 2 hours with the ELISA BrdU label, at a concentration of 10 μM. Following this, cells receive H₂O₂ at a concentration of 3×10²% final volume, or medium alone as a control. Cells are placed in 96-well plates at 2×10⁵ cells/well and incubated at 37° C. At 0 h, 2 h, 4 h and 6 h post-treatment cells are harvested and lysed for ELISA. ELISA was performed on triplicate samples. As seen in FIG. 25 apoptosis in HL-60 cells begins approximately 4 h after treatment with hydrogen peroxide, and the level of apoptosis increases with incubation time. This is in contrast to mock-infected controls that fail to show any signs of apoptotic cell death at any of the indicated time points.

H₂O₂ Induced HL60 Cell Apoptosis is Associated With Significant Changes in Gene Expression.

Cultured HL60 cells are treated with H₂O₂. At various time points post-isolation, total cellular RNA is isolated and examined for gene expression changes using microarray. Significant transcription of mRNA is observed. In one example experiment, as many as 3000 genes are detected using Incyte human ‘LifeGrid’ filters; these genes are increased or decreased by greater than 2-fold over a 2-hour time course as compared with the time zero control HL60 cells.

Many of these regulated genes are ESTs with no known biological functional annotation. Many more have only very little biological characterisation.

Changes in ‘Early’ Gene Expression Include Classes of Genes With ‘Known’ Role in Apoptosis and Genes Associated With Cellular Response to ROS and Oxidative/Environmental Stress.

Of those genes regulated as early as 2 hours post-treatment, classical apoptosis-related genes including both caspase and BCL2 related genes are detected (See Table 8).

Of those genes regulated as early as 2 hours post-treatment, genes are detected that represent the cellular response to oxidative stress (See Table 8).

These observations demonstrate the utility of this model to identify both genes associated with and potentially responsible for the molecular mechanisms of cellular response to ROS and apoptosis.

### EXAMPLE 7

Establishment and Characterisation of a ‘Model Cell-System/Discovery Assay’ to Study the Molecular Mechanisms of Cellular Response to ROS and Apoptosis, in HeLa Cells, by Exogenous Addition of H₂O₂ (‘Induced Apoptosis’).

This example describes the establishment of a model cell system in HeLa cells. The culture of HeLa cells and treatment with exogenous ROS, in the form of H₂O₂ is described. This ‘Model Cell System’ forms the basis of a novel ‘Discovery Assay’ that is then used for the characterisation, identification and functional validation of nucleotide (genes, mRNAs) or polypeptide (proteins, peptides) sequences associated with and potentially responsible for the molecular mechanisms of cellular response to ROS and apoptosis, including; 1) the initiation of the apoptosis process, 2) the cellular signalling pathways, and 3) the cells attempt to combat ROS and apoptosis in the cell.

We demonstrate the utility of this model system to ‘discover’ early regulated genes associated with ROS-induced apoptosis, by the discovery of genes that have ‘known’ involvement in cell apoptosis, carcinogenesis, signal transduction and the cellular response to ROS, oxidative and environmental stress. In many cases these examples include genes, identified by cluster analysis within and between different ROS-induced apoptosis models, that share coordinate regulation and which belong to the same signal transduction pathways or cellular processes. Similarly we demonstrate the identification of genes regulated ‘early’ in ROS-induced apoptosis that have no known biological function i.e. ESTs from both commercial filters and from our own libraries of differentially expressed genes isolated by Suppression Subtractive Hybridisation (SSH). By association with known genes, as described, these novel genes themselves are identified as candidate apoptosis genes that may similarly function in 1) the initiation of the apoptosis process, 2) the cellular signalling pathways, and 3) the cells attempt to combat ROS and apoptosis in the cell.

H₂O₂ Treatment of HeLa Cells Induces Apoptosis

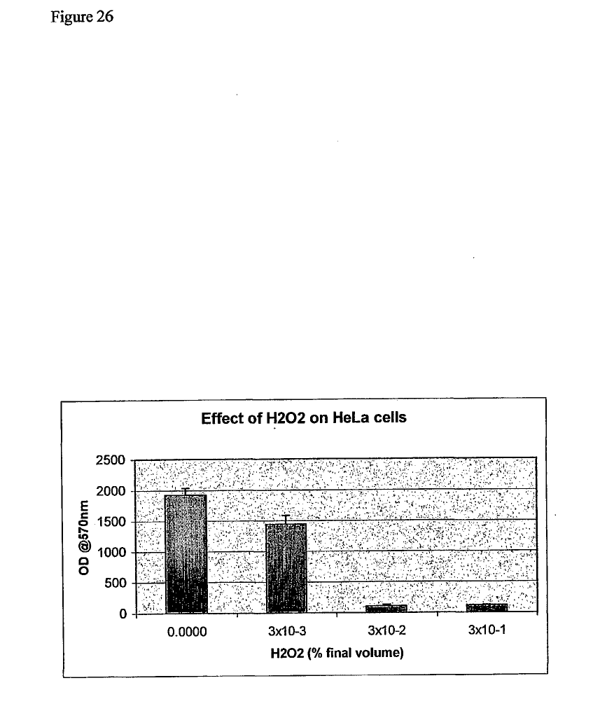

HeLa cells are plated into a 96 well plate (2×10⁵/ml) and allowed to adhere for 4 hours before replacing the medium with fresh culture medium containing the indicated doses of H₂O₂. Cells are cultured for a further 18 h before staining the remaining adherent cells with crystal violet and measuring absorbance at 570 nm. As seen in FIG. 26, the lethal dose of H₂O₂ appeared to occur between 3×10⁻² and 3×10⁻³, at which dose the majority of the cells have undergone apoptosis.

H₂O₂—Induced HeLa Cell Apoptosis is Associated With Significant Changes in Gene Expression.

Cultured HeLa cells are treated with H₂O₂. At various time points post-isolation, total cellular RNA is isolated and examined for gene expression changes using microarray. Significant transcription of mRNA is observed. In one example experiment, as many as 570 genes are detected using Incyte human ‘LifeGrid’ filters; these genes are increased or decreased by greater than 2-fold over a 2-hour time course as compared with the time zero control HeLa cells. Of these regulated-genes, approximately 220 are up-regulated and 350 are down-regulated.

Many of these regulated genes are ESTs with no known biological functional annotation. Many more have only very little biological characterisation.

Changes in ‘Early’ Gene Expression Include Classes of Genes With ‘Known’ Role in Apoptosis and Genes Associated With Cellular Response to ROS and Oxidative/Environmental Stress.

Of those genes regulated as early as 2 hours post-treatment, classical apoptosis-related genes including both caspase and BCL2 related genes are detected (See Table 9).

Of those genes regulated as early as 2 hours post-treatment, genes are detected that represent the cellular response to oxidative stress (See Table 9).

These observations demonstrate the utility of this model to identify both genes associated with and potentially responsible for the molecular mechanisms of cellular response to ROS and apoptosis.

### EXAMPLE 8

Characterisation and Discovery of Oligo/Polynucleotides ‘Genomics’ Associated With Apoptosis

This example describes the characterisation, cloning and analysis of oligonucleotide/polynucleotide sequences whose expression changes are associated with apoptosis. This example also describes the establishment and use of Genomics techniques such as, microarray and subtraction cDNA cloning.

In one example, commercial microarrays are used to measure global gene expression associated with apoptosis. Analysis of such microarray results identifies genes whose expression pattern changes (either up-regulation or down-regulation) in an association with a measurable apoptotic phenotype.

In another example, Suppression Subtractive Hybridisation is used to identify and clone cDNA sequences derived from differentially expressed genes. Such differential gene expression is associated with a measurable apoptotic phenotype. Such cDNA sequences are extended to encompass the full length coding sequence for the mRNA gene product.

Measurement of Global Gene Expression by ‘Microarray’

This example describes the process of microarraying (in the context of a filter based microarraying) and its use to profile gene expression of thousands of genes simultaneously. The microarray process can be separated into three parts: the preparation of the microarray filter, the hybridisation of radiolabelled cDNA probes, and the detection and quantitation of the microarray results.

Construction and Spotting of cDNA Microarray

DNA ‘probe’ sequences are obtained for genes to be represented on a particular microarray. These sequences would typically be EST cDNA sequences cloned in a plasmid vector, either from in-house libraries or obtained from commercial sources (e.g. IMAGE consortium clones). The DNA sequences used to construct the microarray are amplified by PCR using common primer sequences found flanking the multiple cloning sites of most commercial cloning vector plasmids as follows:

M13 (−24) Reverse Primer: 5′ aac agc tat gac cat g 3′

M13 (−48) Reverse Primer: 5′ agc gga taa caa ttt cac aca gga 3′

M13 (−20) Forward Primer: 5′ gta aaa cga cgg cca gt 3′

M13 (−40) Forward Primer: 5′ gtt ttc cca gtc acg ac 3′

T3 Primer: 5′ aat taa ccc tca cta aag gg 3′

T7 Primer: 5′ gta ata cga ctc act ata ggg c 3′

PCR reactions are carried out as follows: 1-2 μl of glycerol stock, miniprep DNA or overnight culture is added to a 20 μl reaction containing, 0.4 μl 10 mM dNTP mix, 1×reaction buffer, 0.4 μl each 20 μM primer and 0.5U Taq polymerase (Qiagen). PCR amplification is carried out with a MWG HT Primus 4×96 well thermocycler as follows: 94° C. 60 s, 35 cycles of 94° C. 40 s, 55° C. 30 s, 72° C. 120 s, followed by 72° C. 120 s.

PCR reaction products are purified using 96 Well PCR Multiscreen (Millipore) as described by the manufacturer.

Purified PCR products are typically eluted in 30-50 μl ddH₂O at a concentration of 50 to 100 ng/μl in 10 mM Tris/lmM EDTA and stored in V-bottom 96-well microtitre plates (Sarstedt).

The arrays are then prepared as follows:

Place a large piece of Whatmann 3MM filter paper into a Pyrex dish and dampen with denaturing solution (1.5M NaCl/0.5N NaOH). In separate dishes place neutralising solution (1M Tris pH7.4/1.5M NaCl) and 2×SSC. Cut a nylon or nitrocellulose filter (for example Hybond-N (APBiotech)) and a piece of Whatmann 3MM paper to the size of a 96-well PCR plate (8cm×12 cm). Place the Nylon filter and Whatmann paper in denaturing solution so that they are damp, but not soaked. Using a tweezers, place both items on a flat support, (Whatmann paper below the filter). Optionally, allocate two wells on the PCR plate for the addition of luciferase (or similar alternative) as a control. The luciferase is added at a concentration of 10 ng/μl. Put the replicator frame in place and taking one plate of PCR products, use the 96-pin replicator to spot the PCR product to designated positions. Place the replicator in alcohol for 2 minutes and then rinse with sterile water. Blot on lint free tissue to dry. Repeat as necessary with additional PCR products. Using the tweezers, transfer the Hybond-N from the support to the dish containing the denaturing solution. Allow it to dampen for 5 minutes. Care must be taken to ensure that the filter isn't soaked in the denaturing solution as diffusion of the DNA may occur. Transfer the Hybond-N to neutralising solution for 5 minutes and then into 2×SSC for a further 2 minutes. Allow the Hybond-N to air dry for at least 15 minutes on a dry piece of Whatmann 3MM paper. Crosslink the cDNA to the Hybond using a UV cross linker (120,000, μJcm-⁻²).

Filter microarrays may also be prepared from PCR reactions using a custom service such as that provided by GeneScreen Ltd (Dorset, UK).

Alternatively, commercial filter arrays ‘Human LifeGrid™’ are purchased from Incyte Genomic Inc. (USA).

Synthesis of Labelled Probes

This example describes the synthesis of a radiolabelled cDNA from total cellular mRNA. The labelled cDNA is used to ‘probe’ DNA fragments, which have been immobilised on to a filter membrane, by complementary hybridisation.

Methodology is as described by manufacturer, for Human LifeGrid™ arrays. Essentially, total cellular RNA (1 μg to 20 μg) or polyA+ mRNA (100 ng to 5 μg) is incubated with an oligo (dT) primer. Primed RNA is reverse transcribed to first stand cDNA in a reaction containing M-MLV reverse transcriptase (RT; alternatively Superscript II is used (Life Sciences)), RT buffer, dNTPs and [α-³³P] dCTP (2000-4000 Ci/mmol) at 42° C. for 1 to 5 h. Unincorporated nucleotides are removed using spin-columns and the labelled probe stored at −80° C. until required.

Labelled probes may also be generated from cDNA, genomic DNA or PCR products. In each case a random primed labelling procedure can be used, for example the Ready-Prime Labelling kit (APBiotech), applied as per manufacturers instructions.

Hybridisation of Filter Based Microarrays

This example describes the complementary hybridisation of radiolabelled cDNA probe to DNA fragments immobilised onto a membrane (typically a nylon or nitrocellulaose filter).

Methodology is as described by manufacturer, for Human LifeGrid™ arrays. Essentially, membrane filters are pre-hybridised in hybridisation buffer (5 to 20 ml) at 42° C. for 2 to 16 h using a hybridisation oven (Hybaid). Following pre-hybridisation, the labelled cDNA probe is added to fresh hybridisation buffer (5 to 20 ml) and hybridised at 42° C. for 14 to 16 h. Following hybridisation, the hybridisation mix is removed and the filters washed with 2×SSC buffer at RT for 5 min., twice with 2×SSC, 1% SDS buffer at 68° C. for 30 min. and twice with 0.6×SSC, 1% SDS buffer at 68° C. for 30 min.

Quantitative Imaging and Analysis of Microarray Filters

This example describes the use of a Phosphoimager to quantitatively image positive signals across the filter arrays.

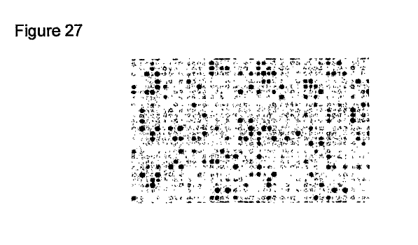

Hybridised filters are wrapped in plastic wrap (Saran) and exposed to a Low-Energy Phosphoimaging screen (Molecular Dynamics). The screen is then placed on the phosphoimager and the gel image captured by scanning at a resolution of 50 microns (See FIG. 27).

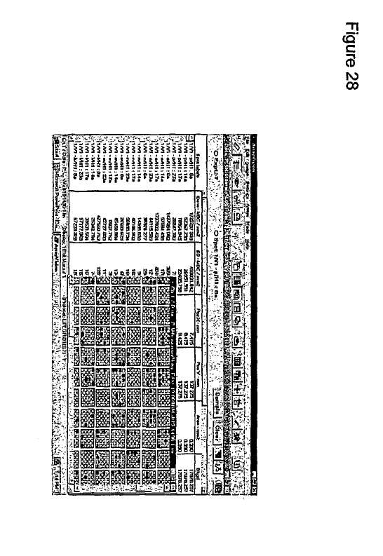

The captured image file is then analysed using software such as Array Vision (Imaging Research Inc.; See FIG. 28). This program contains facilities for spot detection and quantification, and background detection and quantification. This data is then exported to a text file for further analysis. A variety of data fields are exported from the ArrayVision analysis, including; Spot Label, Position, Density, Background, and particularly, Background subtracted density (sDens).

Subsequent analysis may require the calculation of a Normalisation Factor, which facilitates comparison between different experiments. In this example, the Global Mean sDens value is calculated. The Global Mean sDens is calculated as the average of the sDens values across all of the arrays to be compared. A normalisation factor is then derived from division of the overall spot density by the Global mean sDens value. Spot density values (individual sDens) are then corrected by multiplying with the normalisation factor. The data from multiple hybridisation experiments can then be stored in a suitable format, for example in an Access or SQL 7.0 database.

The data from multiple hybridisation experiments can be compared using an analysis and visualisation software facility such as the Cluster and Treeview software (M. Eisen, Stanford Uni, USA).

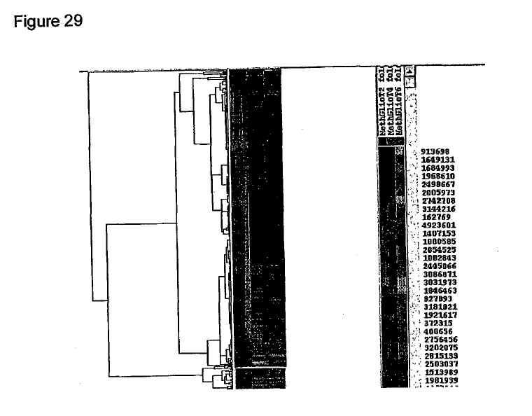

In this example the Cluster software is used to analyse the ‘fold change’ between different arrays derived from hybridising RNA isolated from neutrophils treated with GM-CSF. The Treeview graphical representation of this analysis is shown (See FIG. 29).

The data to be analysed may be imported to the Cluster software as fold change (Tx vs Ty), calculated by comparing the normalised spot density values of Tx with Ty.

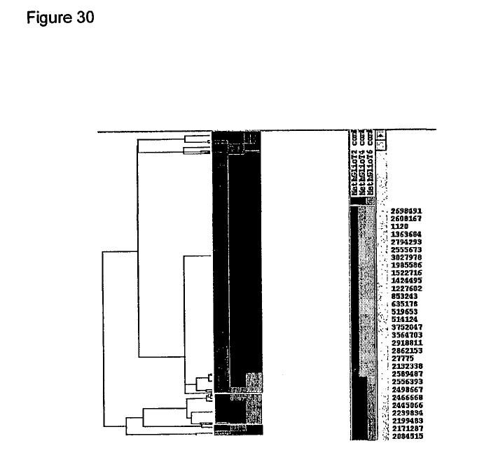

Alternatively, these data may be simplified using codes or combined codes. In this example, each unique gene is represented by at least two identical cDNAs on the array. The fold change value is calculated as described, then for each spot, a value above 5-fold change is accorded a code of 2, a fold-change value of less then 5 but greater then 2 is accorded a code of 1 and a fold-change value of less then 2 is accorded a code value of 0. A combined code is then derived by adding the code values for each identical cDNA on the array. The use of combined codes can greatly simplify the Cluster analysis and the subsequent TreeView visualisation (See FIG. 30).

Suppression Subtractive Hybridisation (SSH) Cloning of Differentially Expressed Genes

This example describes the process of Suppression Subtractive Hybridisation (SSH). SSH is an ‘open’ differential cloning system. Unlike microarray, which requires the analysis of known gene sequences, SSH has the potential to clone all mRNAs that are differentially expressed between a control and test population. We also describe substantial modification to the commercial SSH procedure that enhances its performance.

Total RNA Isolation

Primary or cultured cells are split into control and treatment groups and the treatment group is then challenged with apoptotic stimuli or inhibitors as appropriate. Total RNA is then prepared from both groups using acid phenol/guanidine isothiocyanate extraction (RNAzol B; Biogenesis), or RNeasy RNA preparation kits (Qiagen). Any contaminating genomic DNA is removed by DNase treatment (DNase I, Gibco-BRL).

Extraction of mRNA

The total RNA is then used to prepare mRNA, using the Oligotex mRNA purification kit (Qiagen), or a similar system (Clontech or Strategene). Briefly, mRNA is purified by passing the total RNA over an oligo-dT column. The oligo-dT may be attached to cellulose or glass beads or biotin, and the column may be either spin or gravity format. The bound mRNA is subsequently washed and eluted, ready for use in subtractive hybridisation.

SSH is performed on mRNA purified from treatment or control cells essentially as described (Diatchenko et al 1996), using a PCR-select cDNA subtraction kit (Clontech, K1804). Briefly, cDNA is synthesised from the two pools of mRNA (control and treatment, driver and tester respectively). The resulting cDNA is digested with a restriction enzyme generating a blunt end product (typically Rsal). The tester cDNA is divided into two subsets and distinct adaptor molecules are ligated to each of the cDNA pools. These two samples of tester cDNA are then separately combined with driver cDNA in a solution containing 50 mM HEPES, pH 8.3; 0.5M NaCl; 0.02 mM EDTA, pH 8.0. Following denaturation, (1.5 min, 98° C.), the tester and driver cDNAs are allowed to anneal for 10 hrs at 68° C. After this first hybridisation, the two samples are combined and a fresh portion of heat denatured driver cDNA is added. The samples are allowed to anneal for a further 10 hours at 68° C. The hybridised cDNA is then diluted in a solution containing 20 mM HEPES, pH 8.3; 50 mM NaCI; 0.2 mM EDTA) and heated at 72° C. for 7 minutes, prior to PCR amplification. PCR is performed under standard conditions using the Advantage cDNA PCR kit (Clontech). Only cDNAs that have the correct primer combination, the differentially expressed cDNAs, will amplify exponentially.

Construction of SSH cDNA Libraries

PCR products from the subtractive hybridisation are inserted into a TA cloning vector, for example pCRII T/A cloning kit (Invitrogen). This library of differentially expressed cDNAs is then transformed to Ecoli and the transformants selected for miniprep and sequence analysis.

Plasmid Miniprep

This example describes the extraction of double stranded plasmid DNA from cDNA clones.

Individual colonies (E. coli DH5α or TOP10F′) are picked into 96-deep well culture blocks containing 2.3 ml LB+ampicillin. The culture blocks are shaken at 300 rpm 37° C. for 24 h. Plasmid DNA is isolated using MultiScreen-FB and MultiScreen-NA 96-well plates (Millipore). The methodology is as described by the manufacturer.

DNA Sequencing

This example describes the di-deoxy sequencing of cloned cDNA inserts within the vector pT-Adv (Clontech).

Plasmid miniprep DNA (100 ng to 5 μg) is sent to MWG Biotech for contract sequencing. Sequencing reactions are primed using one of the following universal primer sequences:

M13 (−24) Reverse Primer: 5′ aac agc tat gac cat g 3′

M13 (−48) Reverse Primer: 5′ agc gga taa caa ttt cac aca gga 3′

M13 (−20) Forward Primer: 5′ gta aaa cga cgg cca gt 3′

M13 (−40) Forward Primer: 5′ gtt ttc cca gtc acg ac 3′

T3 Primer: 5′ aat taa ccc tca cta aag gg 3′

T7 Primer: 5′ gta ata cga ctc act ata ggg c 3′

Use of Microarray to Confirm Differential Expression of SSH Clones

The SSH procedure for the cloning of differentially expressed gene products also generates a portion of artifactual cDNAs ‘false positives’ which are not differentially expressed. This example describes our modifications to the commercial SSH, by the use of microarray to confirm the identity of truly differentially expressed clones prior to the laborious task of sequencing.

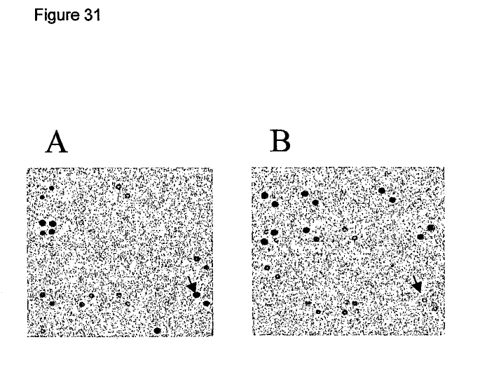

Approximately 1000 colonies are isolated from each reciprocal SSH cDNA library. cDNA insert sequences are amplified by PCR and spotted onto microarray filters as described above. Duplicate filters are hybridised with radio-labelled cDNA probes generated from the reciprocal RNA material used to generate each SSH library (i.e. if an SSH library is made from a subtraction between diseased vs. normal cells, then one filter is hybridised with probe synthesised from RNA isolated from diseased cells and the other from normal cells) i.e. ‘first round of sceening’. An analysis of the filters identifies which cDNA clones are differentially expressed (See FIG. 31).

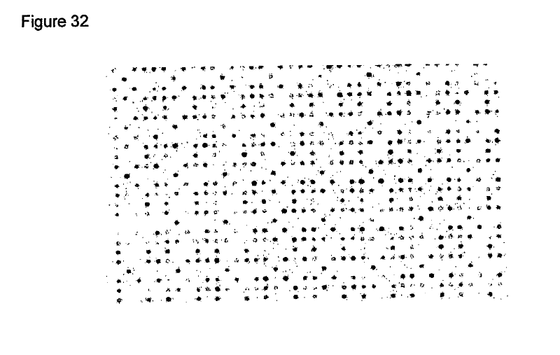

Positive clones from the first round of screening are subsequently submitted to GeneScreen (Dorset, UK) for preparation of a micro array. This array is then hybridised against radiolabelled probes of choice to further analyse the expression profiles of the mRNAs corresponding to the isolated DNA sequences (See FIG. 32).

These arrays may be additionally analysed using the ArrayVision software as described above. The output from this program may be normalised and further analysed using the Cluster and Treeview software, as described above.

Isolation of Full Length Clones for Functional Cloning

DNA sequence for each clone is compared, by homology (BLAST) with public DNA sequence databases (e.g. GENBANK). Where a full length sequence can be identified, flanking PCR primers are designed and the full-length protein coding sequence amplified.

Where the DNA sequence is novel, full length clones are isolated from poly A+ RNA using Smart™ RACE Technology (Clontech) according to the manufacturers protocol. Briefly, double stranded cDNA is generated using the Smart II oligonucleotide and the CDS Primer (both supplied in the kit). Specific PCR products are then generated using the PCR primer (supplied in the kit) and gene specific primers (sense and antisense primers generated from the DNA sequence to be extended). To increase specificity, nested sense and antisense primers are used in secondary PCR amplification. PCR products are then ligated into plasmids, such as the TA cloning system (Invitrogen), transformed into competent cells and expanded. Plasmids are purified using mini-prep isolation system (Qiagen) and plasmids are submitted to MWG for sequencing. Specific 5′ and 3′ sequences are identified using sense and antisense gene specific primers. Products are to be sequenced approximately 10 bases 5′ of the initiation codon for 5′ PCR products and 10 bases 3′ of stop codons for 3′ PCR products. Using sequence data, 5′ and 3′ primers are made and full length cDNA is amplified.

### TABLES

TABLE 1

| | 2 h | 4 h | 6 h | 8 h | 20 h | 24 h |
|---|---|---|---|---|---|---|
| Sample 1 | 6 | 11 | 9 | 44 | 91 | 83 |
| Sample 2 | 7 | 15 | 12 | 48 | — | 93 |

TABLE 2

| Time | 1 h | 2 h | 3 h | 4 h | 5 h | 6 h | 7 h |
|---|---|---|---|---|---|---|---|
| Sample 1 | — | 7.61 | 10.77 | 25.85 | 12.38 | 8.34 | 6.75 |
| Sample 2 | 4.35 | 14.72 | 17.32 | 9.86 | 10.17 | 7.46 | 6.99 |

TABLE 3

| Annotation | 0 h | 2 h | 3 h | 4 h | 5 h | 6 h |
|---|---|---|---|---|---|---|
| caspase 10 (NCBI | 15.44 | 32.4 | 23.2 | 7.46 | 5.82 | 6.09 |
| Unigene ID Hs.5353) | | | | | | |
| BCL2-associated | 0.87 | 1.85 | 0.97 | 0.69 | 0.7 | 0.55 |
| athanogene 5 (Hs.5443) | | | | | | |
| BCL2/adenovirus E1B | | | | | | |
| 19 kD-interacting protein | 16.91 | 12.25 | 15.21 | 29.78 | 37.35 | 44.87 |
| 3-like (Hs.132955) | | | | | | |
| p21 (CDKN1A)-activated | 1.25 | 2.28 | 1.94 | 0.84 | 0.19 | 0.38 |
| kinase 2 (Hs.30692) | | | | | | |
| cyclin-dependent kinase 2 | 1.05 | 2.62 | 2.18 | 1.12 | 2.51 | 2.45 |
| (Hs.19192) | | | | | | |
| v-Ki-ras2 Kirsten rat | 1.04 | 2.54 | 1 | 1.41 | 1.21 | 0.72 |
| sarcoma 2 viral oncogene | | | | | | |
| homolog (Hs.184050) | | | | | | |
| death-associated protein | 0.54 | 1.31 | 0.65 | 0.68 | 0.14 | 0.37 |
| (Hs.75189) | | | | | | |
| death-associated protein | 2.17 | 3.88 | 3.23 | 1.67 | 0.99 | 1.08 |
| kinase 2 (Hs.129208) | | | | | | |
| serine/threonine protein- | 0.63 | 0.51 | 0.77 | 1.21 | 1.84 | 1.66 |
| kinase (Hs.47007) | | | | | | |
| programmed cell death 5 | 1.35 | 1.55 | 1.11 | 0.62 | 0.53 | 0.61 |
| (Hs.166468) | | | | | | |
| absent in melanoma 1 | 0.21 | 0.52 | 0.53 | 0.5 | 0.32 | 0.41 |
| (Hs.161002) | | | | | | |
| tuberous sclerosis 2 | 14.54 | 30.05 | 52.35 | 6.74 | 2.82 | 5.46 |
| (Hs.90303) | | | | | | |
| leukaemia associated | 0.11 | 0.57 | 0.77 | 0.47 | 0.56 | 0.88 |
| gene 1, candidate tumour | | | | | | |
| suppressor (Hs.20149) | | | | | | |
| X-ray repair | 27.84 | 71.45 | 61.04 | 7.45 | 5.02 | 4.71 |
| complementing defective | | | | | | |
| (Hs.129727) | | | | | | |
| Homer, neuronal | 2.12 | 4.37 | 4.28 | 1.77 | 1.71 | 1.34 |
| immediate early gene, 1B | | | | | | |
| (Hs.9192) | | | | | | |
| DNA-damage-inducible | 44.41 | 21.96 | 40.84 | 71.95 | 61.22 | 65.28 |
| transcript 3 (Hs.129913) | | | | | | |
| retinoic acid receptor, | 2.32 | 2.27 | 2.26 | 3.81 | 6.24 | 6.67 |
| beta (Hs.171495) | | | | | | |
| cellular retinoic acid- | 0.66 | 0.74 | 0.81 | 1.09 | 1.19 | 3.95 |
| binding protein 1 | | | | | | |
| (Hs.7678) | | | | | | |
| retinoic acid receptor | 54.4 | 32.12 | 46.54 | 94.87 | 87.76 | 164.75 |
| responder (tazarotene | | | | | | |
| induced) 3 (Hs.17466) | | | | | | |
| runt-related transcription | 37.5 | 9.74 | 29.43 | 33.77 | 39.63 | 49.87 |
| factor 1 (acute myeloid | | | | | | |
| leukaemia 1oncogene | | | | | | |
| Hs. 129914) | | | | | | |
| histone deacetylase 2 | 2.16 | 2.8 | 2.98 | 4.49 | 3.59 | 3.95 |
| (Hs.3352) | | | | | | |
| histone deacetylase 3 | 27.59 | 53.65 | 43.4 | 5.62 | 6.65 | 4.68 |
| (Hs.6975) | | | | | | |
| histone deacetylase 6 | 13.29 | 15.03 | 9.5 | 20.31 | 30.36 | 11.23 |
| (Hs.6764) | | | | | | |
| histone deacetylase 7 | 7.91 | 5.81 | 9.61 | 14.8 | 20.76 | 23.14 |
| (Hs. 116753) | | | | | | |
| hypoxia-inducible factor 1, | 0.98 | 0.7 | 4.02 | 1.82 | 0.51 | 1.72 |
| alpha subunit (Hs.197540) | | | | | | |
| aryl hydrocarbon receptor | 0.39 | 0.38 | 0.17 | 10.29 | 2.5 | 1.12 |
| (Hs.170087) | | | | | | |
| methylmalonate- | 5.2 | 7.56 | 8.49 | 2.69 | 2.24 | 2.5 |
| semialdehyde | | | | | | |
| dehydrogenase | | | | | | |
| (Hs.170008) | | | | | | |
| aldehyde dehydrogenase | 0.91 | 0.58 | 1.37 | 0.96 | 0.24 | 0.77 |
| 3 (Hs.575) | | | | | | |
| aldehyde dehydrogenase | 0.05 | 0.04 | 0.07 | 0.1 | 1.58 | 0.92 |
| 7 (Hs.83155) | | | | | | |
| aldehyde dehydrogenase | 41.49 | 53.26 | 49.09 | 87.32 | 63.84 | 78.1 |
| 5 (Hs.169517) | | | | | | |
| quinone oxidoreductase | 4.53 | 6.47 | 5.28 | 8.57 | 15.54 | 13.81 |
| homolog (Hs.50649) | | | | | | |
| diaphorase | 8.2 | 16.54 | 13.95 | 2.99 | 3.28 | 2.87 |
| (NADH/NADPH) | | | | | | |
| (Hs.80706) | | | | | | |
| UDP glycosyltransferase | 29.19 | 30.2 | 27.1 | 45.99 | 63.05 | 55.11 |
| 8 (Hs.158540) | | | | | | |
| oxygen regulated protein | 1.32 | 1.17 | 1.44 | 1.93 | 7.09 | 4.51 |
| (150 kD; Hs.5417) | | | | | | |
| arachidonate 12- | 0.94 | 0.37 | 1.62 | 1.15 | 8.55 | 8.04 |
| lipoxygenase (Hs.1200) | | | | | | |
| arachidonate 15- | 0.09 | 0.06 | 0.14 | 0.08 | 0.35 | 0.1 |
| lipoxygenase (Hs.73809) | | | | | | |

TABLE 4
a)

| Treatment | 0 hours | 2 hours | 3 hours | 4 hours | 5 hours | 6 hours | 24 hours |
|---|---|---|---|---|---|---|---|
| Neutrophil | 18.87 | 9.25 | 15.24 | 28.4 | 36.56 | 48.61 | |
| spontaneous | | | | | | | |
| apoptosis | | | | | | | |
| UV-irradiation of | 35.44 | 34.93 | | 15.03 | | 19.29 | 13.56 |
| HeLa | | | | | | | |
| Cisplatin treatment | 59.22 | 11.11 | | 17.04 | | | |
| of HeLa | | | | | | | |

b)

| | 0 hours | 2 hours | 3 hours | 4 hours | 6 hours |
|---|---|---|---|---|---|
| Neutrophil spontaneous | 54.4 | 32.12 | 46.54 | 94.87 | |
| apoptosis Expt 1. | | | | | |
| Neutrophil spontaneous | 185.86 | 125.58 | 177.87 | 210.57 | |
| apoptosis Expt 2. | | | | | |
| TNF/CHX treatment of HeLa | 123.39 | 8.76 | | 20.36 | |
| UV-irradiation of HeLa | 113.64 | 135.89 | | 44.39 | 80.07 |
| Cisplatin treatment of HeLa | 166.01 | 50.02 | | 44.55 | |

TABLE 5

| Annotation | 0 hour | 2 hour | 4 hour |
|---|---|---|---|
| caspase 3 (Hs.74552) | 78.18 | 27.72 | 32.46 |
| caspase 6 (Hs.3280) | 0.52 | 0.25 | 0.17 |
| caspase 8 (Hs.19949) | 0.49 | 2.37 | 1.08 |
| caspase 9 (Hs.100641) | 0.29 | 0.77 | 0.7 |
| BCL2/adenovirus E1B 19 kD- | 59.22 | 11.11 | 17.04 |
| interacting protein 3-like | | | |
| (Hs.132955) | | | |
| BCL2 (Hs.76366) | 40.6 | 13.07 | 15.59 |
| BCL2-associated athanogene | 3.62 | 2.02 | 1.65 |
| (Hs.41714) | | | |
| apoptosis inhibitor 4 (survivin; | 86.18 | 26.55 | 26.06 |
| Hs.1578) | | | |
| apoptosis inhibitor 1 (Hs.75263) | 0.19 | 1.29 | 1.09 |
| CASP2 and RIPK1 domain | 40.32 | 17.02 | 12.41 |
| containing adaptor with death | | | |
| domain (Hs.155566) | | | |
| arachidonate 5-lipoxygenase | 0.5 | 1.54 | 1.29 |
| (Hs.89499) | | | |
| arachidonate 12-lipoxygenase, 12R | 2.13E − 09 | 0.47 | 0.4 |
| type (Hs.136574) | | | |
| arachidonate 12-lipoxygenase | 2.01 | 0.33 | 0.54 |
| (Hs.1200) | | | |
| heme oxygenase 1 (Hs.202833) | 1.95 | 4.64 | 3.56 |
| aryl hydrocarbon receptor nuclear | 1.25 | 3.55 | 2.67 |
| translocator (Hs.166172) | | | |
| aryl hydrocarbon receptor nuclear | 2.42 | 5.37 | 3.68 |
| translocator-like (Hs.74515) | | | |
| hypoxia-inducible factor 1, alpha | 2.13E − 09 | 0.9 | 0.82 |
| subunit (Hs.197540) | | | |
| iron-responsive element binding | 0.75 | 1.53 | 0.92 |
| protein 1 (Hs.154721) | | | |

TABLE 6

| UniGene | | Cisplatin | Cisplatin | Cisplatin | | | |
|---|---|---|---|---|---|---|---|
| Cluster | Annotation | T0 | T2 h | T4 h | UV T0 | UV T2 h | UV T4 h |
| Hs.75103 | tyrosine 3/tryptophan | 0.31 | 0.57 | 1.05 | 1.28 | 1.3 | 2.65 |
| | 5-monooxygenase | | | | | | |
| | activation protein | | | | | | |
| Hs.180433 | rTS beta protein | 0.34 | 1.19 | 0.72 | 0.5 | 0.39 | 1.05 |
| | UDP | | | | | | |
| Hs.89691 | glycosyltransferase 2 | 0.94 | 0.64 | 0.43 | 0.67 | 0.46 | 0.28 |
| Hs.132243 | aminopeptidase | 0.78 | 3.28 | 2.88 | 1.97 | 2.47 | 4.23 |
| | puromycin sensitive | | | | | | |
| Hs.79387 | proteasome 26S | 100.88 | 37.19 | 31.78 | 95.11 | 78.39 | 38.86 |
| Hs.178761 | 26S proteasome- | 0.32 | 0.66 | 0.79 | 0.4 | 0.24 | 0.89 |
| | associated pad1 | | | | | | |
| | homolog | | | | | | |
| Hs.81884 | sulfotransferase | 0.48 | 0.52 | 1 | 0.11 | 0.42 | 0.74 |
| | family 2A, | | | | | | |
| | dehydroepiandrostero | | | | | | |
| | ne (DHEA)- | | | | | | |
| | preferring, member 1 | | | | | | |
| Hs.38972 | tetraspan 1 | 0.54 | 3.36 | 4.3 | 1.67 | 2.38 | 5 |

TABLE 7

| Annotation | ggmTzero | T2_ggmTx | T4_ggmTx | T6_ggmTx |
|---|---|---|---|---|
| caspase 3 | 0.64 | 0.45 | 1.32 | 0.7 |
| (Hs.74552) | | | | |
| caspase 9 | 54.91 | 50.34 | 24.55 | 40.68 |
| (Hs.100641) | | | | |
| BCL2/adenovirus | 35.44 | 34.93 | 15.03 | 19.29 |
| E1B 19 kD- | | | | |
| interacting protein | | | | |
| 3-like | | | | |
| (Hs.132955) | | | | |
| BCL2-like 2 | 0.85 | 1.28 | 1.89 | 1.79 |
| (Hs.75244) | | | | |
| CASP2 and | 28.74 | 16.02 | 14.1 | 31.51 |
| RIPK1 domain | | | | |
| containing | | | | |
| adaptor with | | | | |
| death domain | | | | |
| (Hs.155566) | | | | |
| arachidonate 12- | 0.42 | 2.75E − 09 | 4.63E − 09 | 0.36 |
| lipoxygenase, | | | | |
| 12R type | | | | |
| (Hs.136574) | | | | |
| arachidonate 12- | 0.62 | 0.68 | 2.44 | 2.81 |
| lipoxygenase | | | | |
| (Hs.1200) | | | | |
| hypoxia-inducible | 1.43 | 0.48 | 0.000629 | 2.01 |
| factor 1, alpha | | | | |
| subunit | | | | |
| (Hs.197540) | | | | |
| iron-responsive | 45.75 | 39.72 | 14.4 | 40.23 |
| element binding | | | | |
| protein 2 | | | | |
| (Hs.71214) | | | | |

TABLE 8

| Annotation | T 0 h | T 2 h |
|---|---|---|
| caspase 10 (Hs.5353) | 1.19 | 6.71 |
| caspase 8 (Hs.19949) | 0.05 | 1.03 |
| CASP8 and FADD-like | 1.74 | 19.99 |
| apoptosis regulator | | |
| (Hs.195175) | | |
| caspase 10 (Hs.5353) | 1.19 | 6.71 |
| BCL2/adenovirus E1B 19 kD- | 0.00 | 0.32 |
| interacting protein 3-like | | |
| (Hs.132955) | | |
| BCL2-antagonist of cell death | 0.22 | 0.40 |
| (Hs.76366) | | |
| apoptosis inhibitor 4 (survivin) | 0.45 | 1.84 |
| neuronal apoptosis inhibitory | 0.98 | 14.61 |
| protein | | |
| heme oxygenase (decycling) 1 | 0.12 | 0.81 |
| oxygen regulated protein | 0.37 | 1.52 |
| (150 kD) | | |
| arachidonate 15-lipoxygenase | 0.05 | 2.50 |
| aryl hydrocarbon receptor- | 0.12 | 1.35 |
| interacting protein | | |
| aryl hydrocarbon receptor | 0.09 | 0.41 |
| nuclear translocator | | |
| hypoxia-inducible factor 1, | 0.14 | 1.06 |
| alpha subunit (Hs.197540) | | |
| fructose-bisphosphatase 1 | 0.11 | 0.21 |
| (Hs.574) | | |

TABLE 9

| Annotation | T 0 hours | T 2 hours | avg FC |
|---|---|---|---|
| caspase | 2279.334 | 0 | 0.383877027 |
| recruitment | | | |
| domain 4 | | | |
| (Hs.19405) | | | |
| cisplatin | 26.321 | 2681.543 | 2.234528 |
| resistance | | | |
| associated | | | |
| (Hs.166066) | | | |
| arachidonate | 124.849 | 2873.215 | 3.120199 |
| 15- | | | |
| lipoxygenase | | | |
| (Hs.73809) | | | |
| heat shock | 9009.698 | 3924.473 | 0.363199394 |
| 70 kD protein 4 | | | |
| (Hs.90093) | | | |
| heat shock | 1193.491 | 91.82341 | 0.430614336 |
| 70 kD protein 2 | | | |
| (Hs.75452) | | | |
| heat shock | 1520.323 | 617.8009 | 0.367078573 |
| 60 kD protein 1 | | | |
| (Hs.79037) | | | |
| p53-responsive | 2537.969 | 461.2613 | 0.239822085 |
| gene 2 | | | |
| (Hs.118893) | | | |
| hexokinase 4 | 346.283 | 2360.637 | 2.746354 |
| (Hs.89771) | | | |
| hexokinase 2 | 6521.077 | 2728.595 | 0.334782922 |
| (Hs.198427) | | | |

1. A method for identifying a gene product which modulates the transition of a cell between a non-apoptotic state and an apoptotic state, comprising the steps of:
a) determining the level(s) of expression of one or more gene product(s) in a cell to establish a reference expression level;
b) exposing the cell to one or more stimuli which induce the production of intracellular ROS;
c) monitoring the level(s) of expression of said one or more gene product(s) in the cell; and
d) identifying gene product(s) whose expression has been increased, decreased or modified as a result of ROS exposure.

2. A method for identifying a gene product which modulates the transition of a cell between a non-apoptotic state and an apoptotic state, comprising the steps of:
a) determining the level(s) of expression of one or more gene product(s) in a cell to establish a reference expression level;
b) exposing the cell to an inhibitor of apoptosis and/or ROS activity;
c) exposing the cell to one or more stimuli which induce the production of intracellular ROS;
d) monitoring the level(s) of expression of said one or more gene product(s) in the cell; and
e) identifying gene product(s) whose expression has been increased, decreased or modified as a result of ROS exposure in the presence or absence of the apoptosis inhibitor.

3. A method according to claim 1 or claim 2, wherein exposure of the cell to ROS leads to induction of apoptosis in the cell more rapidly than occurs under identical conditions but in the absence of ROS.

4. A method according to any preceding claim, wherein expression levels are determined by assessing polypeptide production.

5. A method according to any one of claims 1 to 4, wherein expression levels are determined by assessing polypeptide post-translational modification.

6. A method according to any one of claims 1 to 5, wherein expression levels are determined by assaying gene transcription.

7. A method according to any preceding claim, wherein the cell is selected from the group consiting of neutrophils, cells with neutrophil characteristics such as HL60 cells, and HeLa cells.

8. A method according to any preceding claim, wherein the ROS is O₂⁻ or H₂O₂.

9. A method according to any preceding claim, wherein the cell is cultured in the presence of an inhibitor of apoptosis, which inhibitor acts to delay the onset of apoptosis in the cell.

10. A method according to claim 9, wherein the inhibitor is GM-CSF.

11. A method according to any preceding claim, wherein the onset of apoptosis is monitored by morphological analysis, externalisation of membrane phospholipid phosphatidyl serine or caspase activation analysis.

12. A method according to claim 11, wherein the involvement of ROS in the induction of the onset of apoptosis is measured by further exposing the cells to one or more ROS inhibitors.

13. A method according to claim 12, wherein the ROS inhibitors are selected from the group consisting of NAC, PTCP and NADPH oxidase inhibitors.

14. A method according to any one of claims 6 to 13, wherein the expression levels of a plurality of gene product(s) are determined by hybridisation of one or more mRNA populations to a set of polynucleotides arrayed on to a substrate.

15. A method according to claim 4 or claim 5, wherein the expression levels of a plurality of gene product(s) are determined by 2D-polyacrylamide gel electrophoresis of one or more polypeptide populations.

16. A method according to any preceding claim, wherein the gene product(s) induce apoptosis in the cell.

17. A method according to any one of claims 1 to 15, wherein the gene product(s) protect the cell against apoptosis.

18. A method according to any preceding claim, wherein the expression level(s) of the gene product(s) are determined by analysis of global gene expression patterns.

19. A method according to claim 18, wherein global gene expression is analysed using microarray technology or SSH.

20. The use of ROS to induce. the expression of gene products which modulate the transition of a cell between an apoptotic state and a non-apoptotic state.

21. A system for modelling ROS-induced apoptosis in a cell, said system comprising:
a) the provision of a population of cells;
b) the exposure of said cell population to one or more stimuli which lead the cell population to undergo ROS-induced apoptosis;
c) optionally, the exposure of said cell population to one or more inhibitors of ROS-induced apoptosis;
d) the analysis of gene expression in the cell population; and
e) the assessment of the onset of apoptosis in said cell population.

22. A system according to claim 21, wherein the cells are selected from the group consisting of primary human neutrophils, HL60 cells and HeLa cells.

23. A system according to claim 20 or claim 21, wherein the stimulus for ROS-induced apoptosis is selected from the group consisting of: exposure to exogenous ROS sources, exposure to cisplatin exposure to UV irradiation and exposure to TNFα and cycloheximide.

24. A system according to claim 20 or 21, wherein the cell is a primary neutrophil and the stimulus for ROS-induced apoptosis is culture of the cell in vitro in a medium.

25. A system according to claim 24, wherein the medium contains serum.

26. A system according to any one of claims 21 to 25, wherein the inhibitor of ROS-induced apoptosis is GM-CSF.

27. A system according to any one of claims 21 to 26, wherein gene expression analysis is performed on a microarray or by SSH.
